SEQUENCE LISTING (43 sequences)

SEQ ID NO: 1 - protein, 10 aa, Artificial Sequence
  Synthetic clone 2155 HCDR3
VGGYYDSMDH

SEQ ID NO: 2 - protein, 9 aa, Artificial Sequence
  Synthetic clone 2155 HCDR2
IISTGGSTY

SEQ ID NO: 3 - protein, 10 aa, Artificial Sequence
  Synthetic clone 2155 HCDR1
GFTISSYAMS

SEQ ID NO: 4 - protein, 9 aa, Artificial Sequence
  Synthetic clone 2155 LCDR3
QQSKEVPWT

SEQ ID NO: 5 - protein, 7 aa, Artificial Sequence
  Synthetic clone 2155 LCDR2
AASNQGS

SEQ ID NO: 6 - protein, 15 aa, Artificial Sequence
  Synthetic clone 2155 LCDR1
RASETVDNYGISFMN

SEQ ID NO: 7 - protein, 118 aa, Artificial Sequence
  Synthetic clone 2155 VH
EVKLVESGGGLVKPGGSLKLSCGASGFTISSYAMSWVRQSPEKRLEWVAIISTGGSTYYPDSVRGRFTISRDNARNSLYLQMSSLRSEDTAMYYCARVGGYYDSMDHWGQGTSVTVSS

SEQ ID NO: 8 - protein, 111 aa, Artificial Sequence
  Synthetic clone 2155 VL
DIVLTQSPASLAVSLGQRATISCRASETVDNYGISFMNWFQQKPGQSPKLLIYAASNQGSGVPARFSGSGSGTDFSLNIHPMEEDDTAMYFCQQSKEVPWTFGGGTKLEIK

SEQ ID NO: 9 - protein, 115 aa, Artificial Sequence
  Synthetic clone 2155 HC1
QVTLVESGGGLVKPGGSLTLSCGASGFTISSYAMSWVRQSPGKALEWVAIISTGGSTYYPDSVRGRFTISRDNAKNSLYLTMSSLDSVDTAMYYCARVGGYYDSMDHWGQGTSVT

SEQ ID NO: 10 - protein, 115 aa, Artificial Sequence
  Synthetic clone 2155 HC2
QVTLVESGGGLVKPGGSLTLSCGASGFTISSYAMSWVRQSPGKALEWVAIISTGGSTYYPDSVRGRFTISRDNAKNSLYLTMSSLDSVDTATYYCARVGGYYDSMDHWGQGTSVT

SEQ ID NO: 11 - protein, 115 aa, Artificial Sequence
  Synthetic clone 2155 HC3a
QVTLVESGGGLVKPGGSLTLSCGASGFTISSYAMSWVRQSPGKALEWVAIISTGGSTYYPDKFRGRFTISRDNAKNSLYLTMSSLRSEDTATYYCARVGGYYDSMDHWGQGTSVT

SEQ ID NO: 12 - protein, 115 aa, Artificial Sequence
  Synthetic clone 2155 HC3b
QVTLKESGGGLVKPGGSLTLSCGASGFTISSYAMSWVRQSPGKALEWVAIISTGGSTYYPDKFRGRFTISRDNAKNSLYLTMSSLRSEDTATYYCARVGGYYDSMDHWGQGTSVT

SEQ ID NO: 13 - protein, 115 aa, Artificial Sequence
  Synthetic clone 2155 HC4
EVQLVESGGGLIQPGGSLKLSCAASGFTISSYAMSWVRQAPGKGLEWVAIISTGGSTYYADSVKGRFTISRDNSKNTLYLQMNSLRAEDTAVYYCARVGGYYDSMDHWGQGTSVT

SEQ ID NO: 14 - protein, 109 aa, Artificial Sequence
  Synthetic clone 2155 LC1
DIVLTQSPASLAASVGDRATISCRASETVDNYGISFMNWFQQKPGKSPKLLIYAASNQGSGVPARFSGSGSGTDFSLNIHPMQPDDTATYFCQQSKEVPWTFGGGTKLE

SEQ ID NO: 15 - protein, 109 aa, Artificial Sequence
  Synthetic clone 2155 LC2a
DIVLTQSPASLSASVGDRATISCRASETVDNYGISFMNWFQQKPGQSPKLLIYAASNQGSGVPARFSGSGSGTDFSLTISPMQPDDTATYYCQQSKEVPWTFGGGTKLE

SEQ ID NO: 16 - protein, 109 aa, Artificial Sequence
  Synthetic clone 2155 LC2b
DIVLTQSPASLSASVGDRATISCRASETVDNYGISYMNWFQQKPGQSPKLLIYAASNQGSGVPARFSGSGSGTDFSLTISPMQPDDTATYYCQQSKEVPWTFGGGTKLE

SEQ ID NO: 17 - protein, 109 aa, Artificial Sequence
  Synthetic clone 2155 LC3
DIVLTQSPASLAVSPGQRATITCRASETVDNYGISFMNWFQQKPGQPPKLLIYAASNQGSGVPARFSGSGSGTDFTLTINPVEADDTANYYCQQSKEVPWTFGQGTKVE

SEQ ID NO: 18 - protein, 9 aa, Artificial Sequence
  Synthetic clone 698 HCDR3, 706 HCDR3
TSTYPHFDY

SEQ ID NO: 19 - protein, 10 aa, Artificial Sequence
  Synthetic clone 698 HCDR2, 706 HCDR2
AIYPGNSDTG

SEQ ID NO: 20 - protein, 10 aa, Artificial Sequence
  Synthetic clone 698 HCDR1, 706 HCDR1
GYSFTTYWMH

SEQ ID NO: 21 - protein, 9 aa, Artificial Sequence
  Synthetic clone 698 LCDR3
QQSNNWPLT

SEQ ID NO: 22 - protein, 8 aa, Artificial Sequence
  Synthetic clone 698 LCDR2, 706 LCDR2, 827 LCDR2, 1649 LCDR2
KYASESIS

SEQ ID NO: 23 - protein, 11 aa, Artificial Sequence
  Synthetic clone 698 LCDR1, 706 LCDR1, 827 LCDR1, 1649 LCDR1
RASQSIGTSIH

SEQ ID NO: 24 - protein, 118 aa, Artificial Sequence
  Synthetic clone 698 VH
EVQLQQSGTVLARPGASVKMSCEASGYSFTTYWMHWIKQRPGQGLEWIGAIYPGNSDTGYNQKFKGKAKLTAVTSATTAYMELSSLTDEDSAVYYCTRTSTYPHFDYWGQGTTLTVSS

SEQ ID NO: 25 - protein, 107 aa, Artificial Sequence
  Synthetic clone 698 VL
DILLTQSPAILSVSPGERVSFSCRASQSIGTSIHWYQQRTNGSPRLLIKYASESISGIPSRFSGSGSGTDFTLNINSVESEDIADYYCQQSNNWPLTFGAGTKLELK

SEQ ID NO: 26 - protein, 9 aa, Artificial Sequence
  Synthetic clone 706 LCDR3, 827 LCDR3, 1649 LCDR3
QQTNNWPLT

SEQ ID NO: 27 - protein, 118 aa, Artificial Sequence
  Synthetic clone 706 VH
EVQLQQSGTVLARPGASVKMSCEASGYSFTTYWMHWIKQRPGQGLEWIGAIYPGNSDTGYNQKFKGKAKLTAVTSASTAYMELSSLTNEDSAVYYCTRTSTYPHFDYWGQGTTLTVSS

SEQ ID NO: 28 - protein, 107 aa, Artificial Sequence
  Synthetic clone 706 VL
DILLTQSPAILSVSPGERVSFSCRASQSIGTSIHWYQQRTNGSPRLLIKYASESISGIPSRFSGSGSGTDFTLNINSVESEDIADYYCQQTNNWPLTFGAGTKLELK

SEQ ID NO: 29 - protein, 9 aa, Artificial Sequence
  Synthetic clone 827 HCDR3, 1649 HCDR3
SSTYPHFDY

SEQ ID NO: 30 - protein, 10 aa, Artificial Sequence
  Synthetic clone 827 HCDR2
TIYPGNSDAG

SEQ ID NO: 31 - protein, 118 aa, Artificial Sequence
  Synthetic clone 827 VH
EVQLQQSGTVLARPGASVKMSCETSGYSFTTYWIHWIKQRPGQGLEWIATIYPGNSDAGYNQKFRGKAKLTAVTSASTAYMELSSLTNEDSAVYYCTRSSTYPHFDYWGQGTTLTVSS

SEQ ID NO: 32 - protein, 107 aa, Artificial Sequence
  Synthetic clone 827 VL, 1649 VL
DILLTQSPAILSVSPGERVSFSCRASQSIGTSIHWYQQRTNDSPRLLIKYASESISGIPSRFSGSGSGTDFTLNINSVESEDIADYYCQQTNNWPLTFGAGTKLELK

SEQ ID NO: 33 - protein, 10 aa, Artificial Sequence
  Synthetic clone 1649 HCDR2
AIYPGNSDAG

SEQ ID NO: 34 - protein, 118 aa, Artificial Sequence
  Synthetic clone 1649 VH
EVQLQQSGTVLAGPGTSVKMSCEASGYSFTTYWIHWIKQRPGQGLEWIGAIYPGNSDAGYNQKFKGKAKLTAVTSASTAYMELSSLTNEDSAVYYCTRSSTYPHFDYWGQGTTLTVSS

SEQ ID NO: 35 - protein, 9 aa, Artificial Sequence
  Synthetic clone 1718 HCDR3
GYGNYYFPY

SEQ ID NO: 36 - protein, 10 aa, Artificial Sequence
  Synthetic clone 1718 HCDR2
WIYPGKGYTN

SEQ ID NO: 37 - protein, 9 aa, Artificial Sequence
  Synthetic clone 1718 HCDR1
DYTFTNYYI

SEQ ID NO: 38 - protein, 9 aa, Artificial Sequence
  Synthetic clone 1718 LCDR3
QQTWSTPWT

SEQ ID NO: 39 - protein, 7 aa, Artificial Sequence
  Synthetic clone 1718 LCDR2
AATSLET

SEQ ID NO: 40 - protein, 11 aa, Artificial Sequence
  Synthetic clone 1718 LCDR1
KASDHIKNWLA

SEQ ID NO: 41 - protein, 118 aa, Artificial Sequence
  Synthetic clone 1718 VH
QVQVQQSGPELVKPGASVRISCKASDYTFTNYYIHWVRQRPGQGLEWLGWIYPGKGYTNYNEKFKGKATLTADKSSSTAYMQFSSLTSEDSAVYFCASGYGNYYFPYWGQGTLVTVSA

SEQ ID NO: 42 - protein, 106 aa, Artificial Sequence
  Synthetic clone 1718 VL
IQMTQSSSYLSVSLGGRVTITCKASDHIKNWLAWYQQKPGNVPRLLMSAATSLETGFPSRFSGSGSGKDFTLTITSLQTEDVATYYCQQYWSTPWTFGGGTKLEIK

SEQ ID NO: 43 - protein, 10 aa, Artificial Sequence
  Synthetic clone 827 HCDR1, 1649 HCDR1
GYSFTTYWIH

(19) United States
(12) Patent Application Publication
(10) Pub. No.: US 2013/0108641 A1
(43) Pub. Date: May 2, 2013

(54) ANTI-GITR ANTIBODIES

(71) Applicants: Nicolas Baurin, Paris (FR); Francis Blanche, Paris (FR); Béatrice Cameron, Paris (FR); Tarik Dabdoubi, Paris (FR); Jeremy Fordham, Bridgewater, NJ (US); Dorothea Kominos, Bridgewater, NJ (US); Jacintha Norohna, Bridgewater, NJ (US); Tim Soos, Bridgewater, NJ (US)

(72) Inventors: Nicolas Baurin, Paris (FR); Francis Blanche, Paris (FR); Béatrice Cameron, Paris (FR); Tarik Dabdoubi, Paris (FR); Jeremy Fordham, Bridgewater, NJ (US); Dorothea Kominos, Bridgewater, NJ (US); Jacintha Norohna, Bridgewater, NJ (US); Tim Soos, Bridgewater, NJ (US)

(73) Assignee: SANOFI, Paris (FR)

(21) Appl. No.: 13/611,789

(22) Filed: Sep. 12, 2012

Related U.S. Application Data
(60) Provisional application No. 61/534,468, filed on Sep. 14, 2011.
(60) Provisional application No. 61/583,306, filed on Jan. 5, 2012.

(30) Foreign Application Priority Data
Jul. 25, 2012 (FR) 1257211

(51) Int. Cl. C07K 16/28; A61K 31/439; A61K 31/56; A61K 38/13; A61K 39/395; A61K 31/519

(52) U.S. Cl. CPC C07K 16/2866; A61K 39/3955; A61K 31/519; A61K 31/56; A61K 38/13; A61K 31/439
USPC 424/139.1; 530/387.9; 536/23.53; 435/320.1; 435/334; 435/69.6

(57) ABSTRACT

The invention provides antibodies that specifically bind to human GITR (hGITR) with high affinity and antagonize the binding of hGITRL to hGITR. The invention also provides pharmaceutical compositions, as well as nucleic acids encoding anti-GITR antibodies, recombinant expression vectors and host cells for making such antibodies, or fragments thereof. Methods of using antibodies of the invention to detect hGITR or to modulate hGITR activity, either in vitro or in vivo, are also provided by the invention.

### RELATED APPLICATIONS

This application claims priority to U.S. Provisional Application No. 61/534,468, filed Sep. 14, 2011, U.S. Provisional Application No. 61/583,306, filed Jan. 5, 2012, and French Patent Application Number 1257211 filed Jul. 25, 2012. The contents of these applications are each hereby incorporated by reference in their entireties.

### BACKGROUND OF THE INVENTION

Glucocorticoid-induced TNFR-related protein (GITR) is expressed in many cells of the innate and adaptive immune system (see, e.g., Hanabuchi et al. (2006) Blood 107:3617-3623; and Nocentini et al. (2005) Eur. J. Immunol. 2005. 35: 1016-1022). GITR cell surface expression is increased following T cell activation and GITR activity modulates effector T lymphocyte and regulatory T cell (Treg) activity (see, e.g., McHugh, et al. (2002) Immunity 2002. 16:311-323; Shimizu, et al. (2002) Nat. Immunol. 3: 135-142; Ronchetti, et al., (2004) Eur. J. Immunol. 34:613-622; and Tone, et al., (2003) Proc. Natl. Acad. Sci. USA 100: 15059-15064).

GITR has also been shown to be expressed in clinical samples collected from patients with inflammatory diseases including: Systemic lupus erythematosus (SLE) (Alunno et al., Reumatismo. 2010 July-September; 62(3):195-201. Italian); Rheumatoid Arthritis (RA) (Mottonen et al., Clin Exp Immunol. 2005 May; 140(2):360-7); and Multiple Sclerosis (MS) (Correale et al., Ann Neurol. 2010 May; 67(5):625-38).

GITR is activated by GITR ligand (GITRL). Loss or block of the GITR/GITRL interaction shows therapeutic benefit in several different murine models of inflammation and immuno-modulation, including models of: lung inflammation (Motta et al., Respir Res. 2009 Oct. 7; 10:93); colitis (Lee et al., Immunology. 2006 December; 119(4):479-87); arthritis (Patel et al., Eur J. Immunol. 2005 December; 35(12):3581-90); MS; Type 1 Diabetes (You et al., PLoS One. 2009 Nov. 20; 4(11):e7848); splanchnic artery occlusion (SAO) shock (Cuzzocrea et al., J Leukoc Biol. 2004 November; 76(5):933-40); and Spinal Cord Injury (Nocentini et al., Mol. Pharmacol. 2008 June; 73(6):1610-21).

Given the link between GITR activity and disease, there is a need in the art for methods and compositions that modulate GITR activity for the treatment of GITR-associated diseases or disorders (e.g., inflammatory disorders).

### SUMMARY OF THE INVENTION

The present invention provides antibodies, or fragments thereof, that specifically bind to human GITR (hGITR) with high affinity and antagonize the binding of hGITRL to hGITR. Such antibodies are particularly useful for treating GITR-associated diseases or disorders (e.g., inflammatory diseases). The invention also provides pharmaceutical compositions, as well as nucleic acids encoding anti-GITR antibodies, recombinant expression vectors and host cells for making such antibodies, or fragments thereof. Methods of using antibodies of the invention to detect human GITR or to modulate human GITR activity, either in vitro or in vivo, are also encompassed by the invention.

Accordingly, in one aspect, the invention provides an isolated monoclonal antibody, or antigen binding portion thereof, that binds specifically to human GITR, the antibody comprising an HCDR3 region amino acid sequence selected from the group consisting of SEQ ID NO: 1, 18, 29, and 35, or conservative amino acid substitutions thereof.

In certain embodiments, the antibody, or antigen binding portion thereof, further comprises an HCDR2 region amino acid sequence selected from the group consisting of SEQ ID NO: 2, 19, 30, 33, and 36, or conservative amino acid substitutions thereof.

In certain embodiments, the antibody, or antigen binding portion thereof, further comprises an HCDR1 region amino acid sequence selected from the group consisting of SEQ ID NO: 3, 20, and 37, or conservative amino acid substitutions thereof.

In certain embodiments, the antibody, or antigen binding portion thereof, further comprises an LCDR3 region amino acid sequence selected from the group consisting of SEQ ID NO: 4, 21, 26, and 38, or conservative amino acid substitutions thereof.

In certain embodiments, the antibody, or antigen binding portion thereof, further comprises an LCDR2 region amino acid sequence selected from the group consisting of SEQ ID NO: 5, 22, and 38, or conservative amino acid substitutions thereof.

In certain embodiments, the antibody, or antigen binding portion thereof, further comprises an LCDR1 region amino acid sequence selected from the group consisting of SEQ ID NO: 6, 23, and 40, or conservative amino acid substitutions thereof.

In another aspect, the invention provides an isolated monoclonal antibody, or antigen binding portion thereof, that binds specifically to human GITR, the antibody comprising an LCDR3 region amino acid sequence selected from the group consisting of SEQ ID NO: 4, 21, 26, and 38, or conservative amino acid substitutions thereof.

In certain embodiments, the antibody, or antigen binding portion thereof, further comprises an LCDR2 region amino acid sequence selected from the group consisting of SEQ ID NO: 5, 22, and 38, or conservative amino acid substitutions thereof.

In certain embodiments, the antibody, or antigen binding portion thereof, further comprises an LCDR1 region amino acid sequence selected from the group consisting of SEQ ID NO: 6, 23, and 40, or conservative amino acid substitutions thereof.

In another aspect, the invention provides an isolated monoclonal antibody, or antigen binding portion thereof, that binds specifically to human GITR, the antibody comprising a heavy chain variable region amino acid sequence with at least 90% identity to an amino acid sequence selected from the group consisting of SEQ ID NOs: 7, 9, 10, 11, 12, 13, 24, 27, 31, 34, and 41.

In certain embodiments, the antibody, or antigen binding portion thereof, further comprises a light chain variable region amino acid sequence with at least 90% identity to an amino acid sequence selected from the group consisting of SEQ ID NOs: 8, 14, 15, 16, 17, 25, 28, 32, and 42.

In another aspect, the invention provides an isolated monoclonal antibody, or antigen binding portion thereof, that binds specifically to human GITR, the antibody comprising a light chain variable region amino acid with at least 90% identity to an amino acid sequence selected from the group consisting of SEQ ID NOs: 8, 14, 15, 16, 17, 25, 28, 32, and 42.

In another aspect, the invention provides an isolated monoclonal antibody, or antigen binding portion thereof, that binds specifically to human GITR, the antibody comprising the heavy chain and light chain variable region amino acid sequences set forth in SEQ ID NO: 7 and 8, SEQ ID NO: 24 and 25, SEQ ID NO: 27 and 28, SEQ ID NO: 31 and 32, SEQ ID NO: 34 and 32, or SEQ ID NO: 41 and 42, respectively.

In another aspect, the invention provides an isolated monoclonal antibody, or antigen binding portion thereof, that binds specifically to human GITR, wherein the antibody, or antigen binding portion thereof, competes for binding to human GITR with an antibody comprising the heavy chain and light chain variable region amino acid sequences set forth in SEQ ID NO: 7 and 8, SEQ ID NO: 24 and 25, SEQ ID NO: 27 and 28, SEQ ID NO: 31 and 32, SEQ ID NO: 34 and 32, or SEQ ID NO: 41 and 42, respectively.

In certain embodiments, the antibodies of the invention further comprise one or more amino acid substitution in the heavy chain variable region at positions selected from the group consisting of H1, H3, H5, H12, H13, H19, H23, H40, H42, H44, H60, H62, H63, H64, H74, H75, H77, H81, H83, H86, H87, H88, and H92.

In certain embodiments, the antibodies of the invention further comprise one or more amino acid substitution in the light chain variable region at positions selected from the group consisting of L12, L13, L15, L17, L22, L36, L46, L47, L76, L78, L80, L82, L83, L84, L89, L91, L104, and L109.

In certain embodiments, the antibodies of the invention bind specifically to cynomolgus monkey GITR.

In another aspect, the invention provides an isolated nucleic acid encoding the amino acid sequence of an antibody, or antigen binding portion thereof, of the invention.

In another aspect, the invention provides a recombinant expression vector comprising a nucleic acid encoding the amino acid sequence of an antibody, or antigen binding portion thereof, of the invention.

In another aspect, the invention provides a host cell containing a recombinant expression vector comprising a nucleic acid encoding the amino acid sequence of an antibody, or antigen binding portion thereof, of the invention.

In another aspect, the invention provides a method of producing an antibody that binds specifically to human GITR, comprising culturing a host cell capable of expressing an antibody of the invention under conditions such that the antibody is produced by the host cell.

In another aspect, the invention provides a pharmaceutical composition comprising an antibody, or antigen binding portion thereof, of the invention and one or more pharmaceutically acceptable carrier.

In another aspect, the invention provides a method for treating a disease or disorder GITR-associated disease or disorder, the method comprising administering to a subject in need of thereof a pharmaceutical composition of the invention.

In certain embodiments, the disease or disorder to be treated is an inflammatory or autoimmune disease or disorder. Suitable diseases include, but are not limited to, chronic obstructive pulmonary disease, systemic lupus erythematosus, rheumatoid arthritis, splanchnic artery occlusion shock, spinal cord injury, type 1 Diabetes, or multiple sclerosis.

In certain embodiments the pharmaceutical composition is administered in combination with one or more additional therapeutic agent. Suitable additional therapeutic agents include, but are not limited to IL-18 antagonists, IL-12 antagonists, TNF antagonists, methotrexate, corticosteroid, cyclosporin, rapamycin, FK506, and non-steroidal anti-inflammatory agents. In a particular embodiment, the additional therapeutic agent(s) is administered concurrently with the pharmaceutical composition of the invention.

### BRIEF DESCRIPTION OF THE DRAWING

FIG. 1 depicts the results of a luciferase reporter assay demonstrating the dose-dependent inhibition of GITR-dependent NFkB signaling by anti-GITR antibody clone 2155.

FIG. 2 depicts the results of a FACS-based assay measuring the binding of anti-GITR antibodies to human PBMCs. M.F.I. denotes means Mean Fluorescence Intensity.

FIG. 3 depicts the results of a FACS-based assay measuring the binding of anti-GITR antibodies to cynomolgus monkey PBMCs. M.F.I. denotes means Mean Fluorescence Intensity.

FIG. 4 depicts the results of an ELISA assay measuring the binding of humanized and chimeric anti-GITR antibodies to hGITR.

FIG. 5 depicts the results of a competition ELISA assay measuring the inhibition of hGITRL binding to hGITR by humanized and chimeric anti-GITR antibodies.

FIG. 6 depicts the results of a FACS-based assay measuring the binding of humanized and chimeric anti-GITR antibodies to HEK293 cells expressing hGITR.

FIG. 7 depicts a Michaelis binding plot of a FACS-based assay measuring the binding of humanized and chimeric anti-GITR antibodies to HEK293 cells expressing hGITR.

### DETAILED DESCRIPTION

The present invention provides antibodies that specifically bind to hGITR. The antibodies of the invention bind to GITR with high affinity and antagonize the binding of hGITRL to hGITR. Such antibodies are particularly useful for treating GITR-associated disease or disorders (e.g., inflammatory diseases). The invention also provides pharmaceutical compositions, as well as nucleic acids encoding anti-GITR antibodies, recombinant expression vectors and host cells for making such antibodies, or fragments thereof. Methods of using antibodies of the invention to detect hGITR or to modulate hGITR activity, either in vitro or in vivo, are also encompassed by the invention.

### I. DEFINITIONS

In order that the present invention may be more readily understood, certain terms are first defined.

As used herein, the term “GITR” refers to “Glucocorticoid-induced TNFR-related protein.” Human and mouse GITR nucleotide and polypeptide sequences are disclosed in WO 98/06842. An exemplary human GITR amino sequence is set forth in GenBank deposit Q9Y5U5/GI:13878830.

As used herein, the term “GITRL” refers to a GITR Ligand protein. Exemplary GITRL proteins include the human GITRL protein set forth in GenBank deposit AAQ89227.1/GI:37182854.

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated V_{H}) and a heavy chain constant region. The heavy chain constant region comprises three domains, C_{H}1, C_{H}2 and C_{H}3. Each light chain comprises a light chain variable region (abbreviated V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

As used herein, the term “antigen-binding portion” of an antibody includes any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as diabodies, triabodies, tetrabodies and minibodies, are also encompassed within the expression “antigen-binding portion.”

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

As used herein the term “framework (FR) amino acid residues” refers to those amino acids in the framework region of an Ig chain. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs.

As used herein, the term “specifically binds to” refers to the ability of an antibody or an antigen-binding fragment thereof to bind to an antigen with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to an antigen with an affinity that is at least two-fold greater than its affinity for a nonspecific antigen.

As used herein, the term “antigen” refers to the binding site or epitope recognized by an antibody or antigen binding portion thereof.

As used herein, the term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms, “plasmid” and “vector” may be used interchangeably. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a recombinant expression vector has been introduced. It should be understood that this term is intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “treat,” “treating,” and “treatment” refer to therapeutic or preventative measures described herein. The methods of “treatment” employ administration to a subject, an antibody or antigen binding portion of the present invention, for example, a subject having a GITR-associated disease or disorder (e.g. an inflammatory disease) or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “GITR-associated disease or disorder” includes disease states and/or symptoms associated with a disease state, where increased levels of GITR and/or activation of cellular signaling pathways involving GITR are found. Exemplary GITR-associated disease or disorder include, but are not limited to, inflammatory diseases.

As used herein, the term “effective amount” refers to that amount of an antibody or an antigen binding portion thereof that binds GITR, which is sufficient to effect treatment, prognosis or diagnosis of a GITR-associated disease or disorder, as described herein, when administered to a subject. A therapeutically effective amount will vary depending upon the subject and disease condition being treated, the weight and age of the subject, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 ug to about 3,500 mg, about 5 ug to about 3,000 mg, about 10 ug to about 2,600 mg, about 20 ug to about 2,575 mg, about 30 ug to about 2,550 mg, about 40 ug to about 2,500 mg, about 50 ug to about 2,475 mg, about 100 ug to about 2,450 mg, about 200 ug to about 2,425 mg, about 300 ug to about 2,000 mg, about 400 ug to about 1,175 mg, about 500 ug to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody or antigen binding portion thereof, according to the invention. Dosage regimens may be adjusted to provide the optimum therapeutic response. An effective amount is also one in which any toxic or detrimental effects (i.e., side effects) of an antibody or antigen binding portion thereof are minimized and/or outweighed by the beneficial effects.

As used herein, the term “subject” includes any human or non-human animal.

As used herein, the term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

As used herein, the term “K_{D}” is intended to refer to the equilibrium dissociation constant of a particular antibody-antigen interaction.

As used herein, the term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain.

### II. Anti-GITR Antibodies

In one aspect the invention provides antibodies, or antigen binding fragments thereof, that specifically bind to hGITR and antagonize the binding of GITRL to GITR. Such antibodies are particularly useful for treating GITR-associated disease or disorders (e.g., inflammatory diseases). Exemplary VH, VL and CDR amino acid sequences of the antibodies of the invention are set forth in Table 1.

TABLE 1
VH, VL and CDR amino acid sequences of exemplary anti-GITR antibodies.

| Antibody Clone | Sequence | SEQ ID NO. |
|---|---|---|
| 2155 HCDR3 | VGGYYDSMDH | 1 |
| 2155 HCDR2 | IISTGGSTY | 2 |
| 2155 HCDR1 | GFTISSYAMS | 3 |
| 2155 LCDR3 | QQSKEVPWT | 4 |
| 2155 LCDR2 | AASNQGS | 5 |
| 2155 LCDR1 | RASETVDNYGISFMN | 6 |
| 2155 VH | EVKLVESGGGLVKPGGSLKLSCGASGFTISSYAM | 7 |
| | SWVRQSPEKRLEWVAIISTGGSTYYPDSVRGRFT | |
| | ISRDNARNSLYLQMSSLRSEDTAMYYCARVGGYY | |
| | DSMDHWGQGTSVTVSS | |
| 2155 VL | DIVLTQSPASLAVSLGQRATISCRASETVDNYGI | 8 |
| | SFMNWFQQKPGQSPKLLIYAASNQGSGVPARFSG | |
| | SGSGTDFSLNIHPMEEDDTAMYFCQQSKEVPWTF | |
| | GGGTKLEIK | |
| 698 HCDR3 | TSTYPHFDY | 18 |
| 698 HCDR2 | AIYPGNSDTG | 19 |
| 698 HCDR1 | GYSFTTYWMH | 20 |
| 698 LCDR3 | QQSNNWPLT | 21 |
| 698 LCDR2 | KYASESIS | 22 |
| 698 LCDR1 | RASQSIGTSIH | 23 |
| 698 VH | EVQLQQSGTVLARPGASVKMSCEASGYSFTTYWM | 24 |
| | HWIKQRPGQGLEWIGAIYPGNSDTGYNQKFKGKA | |
| | KLTAVTSATTAYMELSSLTDEDSAVYYCTRTSTY | |
| | PHFDYWGQGTTLTVSS | |
| 698 VL | DILLTQSPAILSVSPGERVSFSCRASQSIGTSIH | 25 |
| | WYQQRTNGSPRLLIKYASESISGIPSRFSGSGSG | |
| | TDFTLNINSVESEDIADYYCQQSNNWPLTFGAGT | |
| | KLELK | |
| 706 HCDR3 | TSTYPHFDY | 18 |
| 706 HCDR2 | AIYPGNSDTG | 19 |
| 706 HCDR1 | GYSFTTYWMH | 20 |
| 706 LCDR3 | QQTNNWPLT | 26 |
| 706 LCDR2 | KYASESIS | 22 |
| 706 LCDR1 | RASQSIGTSIH | 23 |
| 706 VH | EVQLQQSGTVLARPGASVKMSCEASGYSFTTYWM | 27 |
| | HWIKQRPGQGLEWIGAIYPGNSDTGYNQKFKGKA | |
| | KLTAVTSASTAYMELSSLTNEDSAVYYCTRTSTY | |
| | PHFDYWGQGTTLTVSS | |
| 706 VL | DILLTQSPAILSVSPGERVSFSCRASQSIGTSIH | 28 |
| | WYQQRTNGSPRLLIKYASESISGIPSRFSGSGSG | |
| | TDFTLNINSVESEDIADYYCQQTNNWPLTFGAGT | |
| | KLELK | |
| 827 HCDR3 | SSTYPHFDY | 29 |
| 827 HCDR2 | TIYPGNSDAG | 30 |
| 827 HCDR1 | GYSFTTYWIH | 20 |
| 827 LCDR3 | QQTNNWPLT | 26 |
| 827 LCDR2 | KYASESIS | 22 |
| 827 LCDR1 | RASQSIGTSIH | 23 |
| 827 VH | EVQLQQSGTVLARPGASVKMSCETSGYSFTTYWI | 31 |
| | HWIKQRPGQGLEWIATIYPGNSDAGYNQKFRGKA | |
| | KLTAVTSASTAYMELSSLTNEDSAVYYCTRSSTY | |
| | PHFDYWGQGTTLTVSS | |
| 827 VL | DILLTQSPAILSVSPGERVSFSCRASQSIGTSIH | 32 |
| | WYQQRTNDSPRLLIKYASESISGIPSRFSGSGSG | |
| | TDFTLNINSVESEDIADYYCQQTNNWPLTFGAGT | |
| | KLELK | |
| 1649 HCDR3 | SSTYPHFDY | 29 |
| 1649 HCDR2 | AIYPGNSDAG | 33 |
| 1649 HCDR1 | GYSFTTYWIH | 20 |
| 1649 LCDR3 | QQTNNWPLT | 26 |
| 1649 LCDR2 | KYASESIS | 22 |
| 1649 LCDR1 | RASQSIGTSIH | 23 |
| 1649 VH | EVQLQQSGTVLAGPGTSVKMSCEASGYSFTTYWI | 34 |
| | HWIKQRPGQGLEWIGAIYPGNSDAGYNQKFKGKA | |
| | KLTAVTSASTAYMELSSLTNEDSAVYYCTRSSTY | |
| | PHFDYWGQGTTLTVSS | |
| 1649 VL | DILLTQSPAILSVSPGERVSFSCRASQSIGTSIH | 32 |
| | WYQQRTNDSPRLLIKYASESISGIPSRFSGSGSG | |
| | TDFTLNINSVESEDIADYYCQQTNNWPLTFGAGT | |
| | KLELK | |
| 1718 HCDR3 | GYGNYYFPY | 35 |
| 1718 HCDR2 | WIYPGKGYTN | 36 |
| 1718 HCDR1 | DYTFTNYYI | 37 |
| 1718 LCDR3 | QQTWSTPWT | 38 |
| 1718 LCDR2 | AATSLET | 39 |
| 1718 LCDR1 | KASDHIKNWLA | 40 |
| 1718 VH | QVQVQQSGPELVKPGASVRISCKASDYTFTNYYI | 41 |
| | HWVRQRPGQGLEWLGWIYPGKGYTNYNEKFKGKA | |
| | TLTADKSSSTAYMQFSSLTSEDSAVYFCASGYGN | |
| | YYFPYWGQGTLVTVSA | |
| 1718 VL | IQMTQSSSYLSVSLGGRVTITCKASDHIKNWLAW | 42 |
| | YQQKPGNVPRLLMSAATSLETGFPSRFSGSGSGK | |
| | DFTLTITSLQTEDVATYYCQQYWSTPWTFGGGTK | |
| | LEIK | |

In certain embodiments, the antibody, or antigen binding fragment thereof, comprises one or more CDR region amino acid sequences selected from the group consisting of SEQ ID NO: 1, 2, 3, 4, 5, 6, 18, 19, 20, 21, 22, 23, 26, 29, 33, 25, 26, 37, 38, 39, and 40.

In other embodiments, the antibody, or antigen binding fragment thereof, comprises HCDR3, HCDR2 and HCDR1 region amino acid sequences selected from the group consisting of:

a) SEQ ID NO: 1, 2 and 3;

b) SEQ ID NO: 18, 19 and 20;

c) SEQ ID NO: 29, 30 and 20;

d) SEQ ID NO: 29, 33 and 20;

e) SEQ ID NO: 29, 33 and 20; and

f) SEQ ID NO: 35, 36 and 37, respectively.

In other embodiments, the antibody, or antigen binding fragment thereof, comprises the LCDR3, LCDR2 and LCDR1 region amino acid sequences selected from the group consisting of:

a) SEQ ID NO: 4, 5 and 6;

b) SEQ ID NO: 21, 22 and 23;

c) SEQ ID NO: 26, 22 and 23; and

d) SEQ ID NO: 38, 39 and 40, respectively.

In other embodiments, the antibody, or antigen binding fragment thereof, comprises the HCDR3, HCDR2, HCDR1, LCDR3, LCDR2 and LCDR1 region amino acid sequences selected from the group consisting of:

a) SEQ ID NO: 1, 2, 3, 4, 5 and 6;

b) SEQ ID NO: 18, 19, 20, 21, 22 and 23;

c) SEQ ID NO: 18, 19, 20, 26, 22 and 23;

d) SEQ ID NO: 29, 30, 20, 26, 22 and 23;

e) SEQ ID NO: 29, 33, 20, 26, 22 and 23; and

f) SEQ ID NO: 35, 36, 37, 38, 39 and 40, respectively

In other embodiments, the antibody, or antigen binding fragment thereof, comprises the VH region amino acid sequences set forth in SEQ ID NO: 7, 24, 27, 31, 34, and/or 41.

In other embodiments, the antibody, or antigen binding fragment thereof, comprises the VL region amino acid sequences set forth in SEQ ID NO: 8, 25, 28, 32, and/or 42.

In other embodiments, the antibody, or antigen binding fragment thereof, comprises the VH and VL region amino acid sequences selected from the group consisting of: SEQ ID NO: 7 and 8; SEQ ID NO: 24 and 25; SEQ ID NO: 27 and 28; SEQ ID NO: 31 and 32; SEQ ID NO: 34 and 32; and SEQ ID NO: 41 and 42, respectively.

In certain embodiments, the antibody, or antigen binding fragment thereof, comprises one or more CDR region amino acid sequence selected from the group consisting of SEQ ID NO: 1, 2, 3, 4, 5, 6, 18, 19, 20, 21, 22, 23, 26, 29, 33, 25, 26, 37, 38, 39, and 40, wherein the one or more CDR region amino acid sequences comprises at least one or more conservative amino acid substitutions.

The present invention also encompasses “conservative amino acid substitutions” in the CDR amino acid sequences (e.g., SEQ ID NOs: 1, 2, 3, 4, 5, 6, 18, 19, 20, 21, 22, 23, 26, 29, 33, 25, 26, 37, 38, 39, and 40) of the antibodies of the invention, i.e., amino acid sequence modifications which do not abrogate the binding of the antibody to the antigen, e.g., GITR. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-GITR antibody is preferably replaced with another amino acid residue from the same class. Methods of identifying amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

In another embodiment, the present invention provides anti-GITR antibodies, or antigen binding fragment thereof, that comprise a VH and/or VL region amino acid sequence with about 80%, 85%, 86%, 87%, 88%, 89%, 90%, 91%, 92%, 93%, 94%, 95%, 96%, 97%, 98%, or 99%, identity to the VH region amino acid sequence set forth in SEQ ID NO: 7, 9, 10, 11, 12, 13, 24, 27, 31, 34, or 41, or the VL region amino acid sequence set forth in SEQ ID NO: 8, 14, 15, 16, 17, 24, 27, 31, 34, or 41, respectively.

In another embodiment, the present invention provides anti-GITR antibodies that bind to the same epitope and/or cross compete with an antibody, or antigen binding fragment thereof comprising the VH and VL region amino acid sequences set forth in SEQ ID NO: 7 and 8, respectively. Such antibodies can be identified using routine competition binding assays including, for example, surface plasmon resonance (SPR)-based competition assays.

In another embodiment, the present invention provides anti-GITR antibodies that bind to cell surface GITR with a Kd of at least about 1×10⁻⁹ M.

In another embodiment, the present invention provides anti-GITR antibodies that bind to GITR and block the binding of GITRL to GITR with an IC50 of less than 1×10⁻⁹ M.

### III. HUMANIZED ANTI-GITR ANTIBODIES

In one aspect, the invention provides humanized anti-GITR antibodies, or antigen binding fragments thereof, comprising one or more CDR regions (or conservatively modified variants thereof) from the murine anti-GITR antibody, 2155, disclosed herein.

Any method of humanization can be employed to generate the humanized anti-GITR antibodies of the invention. Suitable methods are disclosed herein and specifically exemplified in Example 7. The VH and VL amino acid sequences of exemplary humanized VH and VL region amino acid sequences are set forth in Table 2.

TABLE 2
VH and VL amino acid sequences of exemplary
humanized anti-GITR antibodies.

| VH or | | SEQ |
|---|---|---|
| VL variant | Sequence | ID NO. |
| clone 2155 | QVTLVESGGGLVKPGGSLTLSCGASGFTIS | 9 |
| HC1 | SYAMSWVRQSPGKALEWVAIISTGGSTYYP | |
| | DSVRGRFTISRDNAKNSLYLTMSSLDSVDT | |
| | AMYYCARVGGYYDSMDHWGQGTSVT | |
| clone 2155 | QVTLVESGGGLVKPGGSLTLSCGASGFTIS | 10 |
| HC2 | SYAMSWVRQSPGKALEWVAIISTGGSTYYP | |
| | DSVRGRFTISRDNAKNSLYLTMSSLDSVDT | |
| | ATYYCARVGGYYDSMDHWGQGTSVT | |
| clone 2155 | QVTLVESGGGLVKPGGSLTLSCGASGFTIS | 11 |
| HC3a | SYAMSWVRQSPGKALEWVAIISTGGSTYYP | |
| | DKFRGRFTISRDNAKNSLYLTMSSLRSEDT | |
| | ATYYCARVGGYYDSMDHWGQGTSVT | |
| clone 2155 | QVTLKESGGGLVKPGGSLTLSCGASGFTIS | 12 |
| HC3b | SYAMSWVRQSPGKALEWVAIISTGGSTYYP | |
| | DKFRGRFTISRDNAKNSLYLTMSSLRSEDT | |
| | ATYYCARVGGYYDSMDHWGQGTSVT | |
| clone 2155 | EVQLVESGGGLIQPGGSLKLSCAASGFTIS | 13 |
| HC4 | SYAMSWVRQAPGKGLEWVAIISTGGSTYYA | |
| | DSVKGRFTISRDNSKNTLYLQMNSLRAEDT | |
| | AVYYCARVGGYYDSMDHWGQGTSVT | |
| clone 2155 | DIVLTQSPASLAASVGDRATISCRASETVD | 14 |
| LC1 | NYGISFMNWFQQKPGKSPKLLIYAASNQGS | |
| | GVPARFSGSGSGTDFSLNIHPMQPDDTATY | |
| | FCQQSKEVPWTFGGGTKLE | |
| clone 2155 | DIVLTQSPASLSASVGDRATISCRASETVD | 15 |
| LC2a | NYGISFMNWFQQKPGQSPKLLIYAASNQGS | |
| | GVPARFSGSGSGTDFSLTISPMQPDDTATY | |
| | YCQQSKEVPWTFGGGTKLE | |
| clone 2155 | DIVLTQSPASLSASVGDRATISCRASETVD | 16 |
| LC2b | NYGISYMNWFQQKPGQSPKLLIYAASNQGS | |
| | GVPARFSGSGSGTDFSLTISPMQPDDTATY | |
| | YCQQSKEVPWTFGGGTKLE | |
| clone 2155 | DIVLTQSPASLAVSPGQRATITCRASETVD | 17 |
| LC3 | NYGISFMNWFQQKPGQPPKLLIYAASNQGS | |
| | GVPARFSGSGSGTDFTLTINPVEADDTANY | |
| | YCQQSKEVPWTFGQGTKVE | |

In certain embodiments, the humanized anti-GITR antibody, or antigen binding portion thereof, comprises the VH region amino acid sequence set forth in SEQ ID NO: 7. with one or more amino acid substitution(s) at positions selected from the group consisting of H1, H3, H5, H12, H13, H19, H23, H40, H42, H44, H60, H62, H63, H64, H74, H75, H77, H81, H83, H86, H87, H88, and H92.

In a one particular embodiment, the humanized anti-GITR antibody, or antigen binding portion thereof comprises the VH region amino acid sequence set forth in SEQ ID NO: 9, 10, 11, 12, or 13.

In certain embodiment, the humanized anti-GITR antibody, or antigen binding portion thereof, comprises the VL region amino acid sequences set forth in SEQ ID NO: 8. with one or more amino acid substitution(s) at positions selected from the group consisting of L12, L13, L15, L17, L22, L36, L46, L47, L76, L78, L80, L82, L83, L84, L89, L91, L104, and L109.

In a one particular embodiment, the humanized anti-GITR antibody, or antigen binding portion thereof comprises the VL region amino acid sequence set forth in SEQ ID NO: 14, 15, 16, or 17.

In another particular embodiment, the humanized anti-GITR antibody, or antigen binding portion thereof comprises the VH and VL region amino acid sequence set forth in: SEQ ID NO: 9 and 14; SEQ ID NO: 10 and 14; SEQ ID NO: 11 and 15; SEQ ID NO: 12 and 15; or SEQ ID NO: 13 and 17, respectively.

### IV. MODIFIED ANTI-GITR ANTIBODIES

In certain embodiments, anti-GITR antibodies of the invention may comprise one or more modifications. Modified forms of anti-GITR antibodies of the invention can be made using any techniques known in the art.

### i) Reducing Immunogenicity

In certain embodiments, anti-GITR antibodies, or antigen binding fragments thereof, of the invention are modified to reduce their immunogenicity using art-recognized techniques. For example, antibodies, or fragments thereof, can be chimerized, humanized, and/or deimmunized.

In one embodiment, an antibody, or antigen binding fragments thereof, of the invention may be chimeric. A chimeric antibody is an antibody in which different portions of the antibody are derived from different animal species, such as antibodies having a variable region derived from a murine monoclonal antibody and a human immunoglobulin constant region. Methods for producing chimeric antibodies, or fragments thereof, are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference herein in their entireties. Techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) may be employed for the synthesis of said molecules. For example, a genetic sequence encoding a binding specificity of a mouse anti-GITR antibody molecule may be fused together with a sequence from a human antibody molecule of appropriate biological activity. As used herein, a chimeric antibody is a molecule in which different portions are derived from different animal species, such as those having a variable region derived from a murine monoclonal antibody and a human immunoglobulin constant region, e.g., humanized antibodies.

In another embodiment, an antibody, or antigen binding portion thereof, of the invention is humanized. Humanized antibodies, have a binding specificity comprising one or more complementarity determining regions (CDRs) from a non-human antibody and framework regions from a human antibody molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Reichmann et al., Nature 332:323 (1988), which are incorporated herein by reference herein in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

In a particular embodiment, a humanization method is employed that is based on the impact of the molecular flexibility of the antibody during and at immune recognition. Protein flexibility is related to the molecular motion of the protein molecule. Protein flexibility is the ability of a whole protein, a part of a protein or a single amino acid residue to adopt an ensemble of conformations which differ significantly from each other. Information about protein flexibility can be obtained by performing protein X-ray crystallography experiments (see, for example, Kundu et al. 2002, Biophys J 83:723-732.), nuclear magnetic resonance experiments (see, for example, Freedberg et al., J Am Chem Soc 1998, 120(31):7916-7923) or by running molecular dynamics (MD) simulations. An MD simulation of a protein is done on a computer and allows one to determine the motion of all protein atoms over a period of time by calculating the physical interactions of the atoms with each other. The output of a MD simulation is the trajectory of the studied protein over the period of time of the simulation. The trajectory is an ensemble of protein conformations, also called snapshots, which are periodically sampled over the period of the simulation, e.g. every 1 picosecond (ps). It is by analyzing the ensemble of snapshots that one can quantify the flexibility of the protein amino acid residues. Thus, a flexible residue is one which adopts an ensemble of different conformations in the context of the polypeptide within which that residue resides. MD methods are known in the art, see, e.g., Brooks et al. “Proteins: A Theoretical Perspective of Dynamics, Structure and Thermodynamics” (Wiley, New York, 1988). Several software enable MD simulations, such as Amber (see Case et al. (2005) J Comp Chem 26:1668-1688), Charmm (see Brooks et al. (1983) J Comp Chem 4:187-217; and MacKerell et al. (1998) in “The Encyclopedia of Computational Chemistry” vol. 1:271-177, Schleyer et al., eds. Chichester: John Wiley & Sons) or Impact (see Rizzo et al. J Am Chem Soc; 2000; 122(51):12898-12900.)

Most protein complexes share a relatively large and planar buried surface and it has been shown that flexibility of binding partners provides the origin for their plasticity, enabling them to conformationally adapt to each other (Structure (2000) 8, R137-R142). As such, examples of “induced fit” have been shown to play a dominant role in protein-protein interfaces. In addition, there is a steadily increasing body of data showing that proteins actually bind ligands of diverse shapes sizes and composition (Protein Science (2002) 11:184-187) and that the conformational diversity appears to be an essential component of the ability to recognize different partners (Science (2003) 299, 1362-1367). Flexible residues are involved in the binding of protein-protein partners (Structure (2006) 14, 683-693).

The flexible residues can adopt a variety of conformations that provide an ensemble of interaction areas that are likely to be recognized by memory B cells and to trigger an immunogenic response. Thus, an antibody can be humanized by modifying a number of residues from the framework so that the ensemble of conformations and of recognition areas displayed by the modified antibody resemble as much as possible those adopted by a human antibody. That can be achieved by modifying a limited number of residues by: (1) building a homology model of the parent mAb and running an MD simulation; (2) analyzing the flexible residues and identification of the most flexible residues of a non-human antibody molecule, as well as identifying residues or motifs likely to be a source of heterogeneity or of degradation reaction; (3) identifying a human antibody which displays the most similar ensemble of recognition areas as the parent antibody; (4) determining the flexible residues to be mutated, residues or motifs likely to be a source of heterogeneity and degradation are also mutated; and (5) checking for the presence of known T cell or B cell epitopes. The flexible residues can be found using an MD calculation as taught herein using an implicit solvent model, which accounts for the interaction of the water solvent with the protein atoms over the period of time of the simulation.

Once the set of flexible residues has been identified within the variable light and heavy chains, a set of human heavy and light chain variable region frameworks that closely resemble that of the antibody of interest are identified. That can be done, for example, using a BLAST search on the set of flexible residues against a database of antibody human germ line sequence. It can also be done by comparing the dynamics of the parent mAb with the dynamics of a library of germ line canonical structures. The CDR residues and neighboring residues are excluded from the search to ensure high affinity for the antigen is preserved. Flexible residues then are replaced.

When several human residues show similar homologies, the selection is driven also by the nature of the residues that are likely to affect the solution behavior of the humanized antibody. For instance, polar residues will be preferred in exposed flexible loops over hydrophobic residues. Residues which are a potential source of instability and heterogeneity are also mutated even if there are found in the CDRs. That will include exposed methionines as sulfoxide formation can result from oxygen radicals, proteolytic cleavage of acid labile bonds such as those of the Asp-Pro dipeptide (Drug Dev Res (2004) 61:137-154), deamidation sites found with an exposed asparagine residue followed by a small amino acid, such as Gly, Ser, Ala, His, Asn or Cys (J Chromatog (2006) 837:35-43) and N-glycosylation sites, such as the Asn-X-Ser/Thr site. Typically, exposed methionines will be substituted by a Leu, exposed asparagines will be replaced by a glutamine or by an aspartate, or the subsequent residue will be changed. For the glycosylation site (Asn-X-Ser/Thr), either the Asn or the Ser/Thr residue will be changed.

The resulting composite antibody sequence is checked for the presence of known B cell or linear T-cell epitopes. A search is performed, for example, with the publicly available Immune Epitope Data Base (IEDB) (PLos Biol (2005) 3(3)e91). If a known epitope is found within the composite sequence, another set of human sequences is retrieved and substituted. Thus, unlike the resurfacing method of U.S. Pat. No. 5,639,641, both B-cell-mediated and T-cell-mediated immunogenic responses are addressed by the method. The method also avoids the issue of loss of activity that is sometimes observed with CDR grafting (U.S. Pat. No. 5,530,101). In addition, stability and solubility issues also are considered in the engineering and selection process, resulting in an antibody that is optimized for low immunogenicity, high antigen affinity and improved biophysical properties.

In some embodiments, de-immunization can be used to decrease the immunogenicity of and antibody, or antigen binding portion thereof. As used herein, the term “de-immunization” includes alteration of an antibody, or antigen binding portion thereof, to modify T cell epitopes (see, e.g., WO9852976A1, WO0034317A2). For example, VH and VL sequences from the starting antibody may be analyzed and a human T cell epitope “map” may be generated from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of GITR-specific antibodies or fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

### ii) Effector Functions and Fc Modifications

Anti-GITR antibodies of the invention may comprise an antibody constant region (e.g. an IgG constant region e.g., a human IgG constant region, e.g., a human IgG1 or IgG4 constant region) which mediates one or more effector functions. For example, binding of the C1 component of complement to an antibody constant region may activate the complement system. Activation of complement is important in the opsonization and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. In preferred embodiments, the antibodies, or fragments thereof, of the invention bind to an Fc.gamma receptor. In alternative embodiments, anti-GITR antibodies of the invention may comprise a constant region which is devoid of one or more effector functions (e.g., ADCC activity) and/or is unable to bind Fcγ receptor.

Certain embodiments of the invention include anti-GITR antibodies in which at least one amino acid in one or more of the constant region domains has been deleted or otherwise altered so as to provide desired biochemical characteristics such as reduced or enhanced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies, or fragments thereof, for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain other embodiments, anti-GITR antibodies comprise constant regions derived from different antibody isotypes (e.g., constant regions from two or more of a human IgG1, IgG2, IgG3, or IgG4). In other embodiments, anti-GITR antibodies comprises a chimeric hinge (i.e., a hinge comprising hinge portions derived from hinge domains of different antibody isotypes, e.g., an upper hinge domain from an IgG4 molecule and an IgG1 middle hinge domain). In one embodiment, an anti-GITR antibody comprises an Fc region or portion thereof from a human IgG4 molecule and a Ser228Pro mutation (EU numbering) in the core hinge region of the molecule.

In certain anti-GITR antibodies, the Fc portion may be mutated to increase or decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or flexibility. The resulting physiological profile, bioavailability and other biochemical effects of the modifications, such as tumor localization, biodistribution and serum half-life, may easily be measured and quantified using well know immunological techniques without undue experimentation.

In certain embodiments, an Fc domain employed in an antibody of the invention is an Fc variant. As used herein, the term “Fc variant” refers to an Fc domain having at least one amino acid substitution relative to the wild-type Fc domain from which said Fc domain is derived. For example, wherein the Fc domain is derived from a human IgG1 antibody, the Fc variant of said human IgG1 Fc domain comprises at least one amino acid substitution relative to said Fc domain.

The amino acid substitution(s) of an Fc variant may be located at any position (i.e., any EU convention amino acid position) within the Fc domain. In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

The antibodies of the invention may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, and WO06/085967A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated by reference herein. In one exemplary embodiment, an antibody of the invention may comprise an Fc variant comprising an amino acid substitution at EU position 268 (e.g., H268D or H268E). In another exemplary embodiment, an antibody of the invention may comprise an amino acid substitution at EU position 239 (e.g., S239D or S239E) and/or EU position 332 (e.g., I332D or I332Q).

In certain embodiments, an antibody of the invention may comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization to the brain, kidney, and/or liver is desired. In one exemplary embodiment, the altered antibodies of the invention exhibit reduced transport across the epithelium of kidney glomeruli from the vasculature. In another embodiment, the altered antibodies of the invention exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions which altered FcRn binding activity are disclosed in International PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In other embodiments, antibodies, for use in the diagnostic and treatment methods described herein have a constant region, e.g., an IgG1 or IgG4 heavy chain constant region, which is altered to reduce or eliminate glycosylation. For example, an antibody of the invention may also comprise an Fc variant comprising an amino acid substitution which alters the glycosylation of the antibody. For example, said Fc variant may have reduced glycosylation (e.g., N- or O-linked glycosylation). In exemplary embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In a particular embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduced or altered glycosylation are disclosed in International PCT Publication No. WO05/018572, which is incorporated by reference herein. In preferred embodiments, the antibodies, or fragments thereof, of the invention are modified to eliminate glycosylation. Such antibodies, or fragments thereof, may be referred to as “agly” antibodies, or fragments thereof, (e.g. “agly” antibodies). While not being bound by theory, it is believed that “agly” antibodies, or fragments thereof, may have an improved safety and stability profile in vivo. Exemplary agly antibodies, or fragments thereof, comprise an aglycosylated Fc region of an IgG4 antibody which is devoid of Fc-effector function thereby eliminating the potential for Fc mediated toxicity to the normal vital organs that express GITR. In yet other embodiments, antibodies, or fragments thereof, of the invention comprise an altered glycan. For example, the antibody may have a reduced number of fucose residues on an N-glycan at Asn297 of the Fc region, i.e., is afucosylated. In another embodiment, the antibody may have an altered number of sialic acid residues on the N-glycan at Asn297 of the Fc region.

iii) Covalent Attachment

Anti-GITR antibodies of the invention may be modified, e.g., by the covalent attachment of a molecule to the antibody such that covalent attachment does not prevent the antibody from specifically binding to its cognate epitope. For example, but not by way of limitation, the antibodies, or fragments thereof, of the invention may be modified by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the derivative may contain one or more non-classical amino acids.

Antibodies, or fragments thereof, of the invention may further be recombinantly fused to a heterologous polypeptide at the N- or C-terminus or chemically conjugated (including covalent and non-covalent conjugations) to polypeptides or other compositions. For example, anti-GITR antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Anti-GITR antibodies may be fused to heterologous polypeptides to increase the in vivo half life or for use in immunoassays using methods known in the art. For example, in one embodiment, PEG can be conjugated to the anti-GITR antibodies of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, anti-GITR antibodies of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Anti-GITR antibodies of the invention may be used in non-conjugated form or may be conjugated to at least one of a variety of molecules, e.g., to improve the therapeutic properties of the molecule, to facilitate target detection, or for imaging or therapy of the patient. Anti-GITR antibodies of the invention can be labeled or conjugated either before or after purification, when purification is performed. In particular, anti-GITR antibodies of the invention may be conjugated to therapeutic agents, prodrugs, peptides, proteins, enzymes, viruses, lipids, biological response modifiers, pharmaceutical agents, or PEG.

The present invention further encompasses anti-GITR antibodies of the invention conjugated to a diagnostic or therapeutic agent. The anti-GITR antibodies can be used diagnostically to, for example, monitor the development or progression of a immune cell disorder (e.g., CLL) as part of a clinical testing procedure to, e.g., determine the efficacy of a given treatment and/or prevention regimen. Detection can be facilitated by coupling the anti-GITR antibodies to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, ¹³¹I, 111In or 99Tc.

Anti-GITR antibodies for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG.

In another embodiment, an anti-GITR antibody for use in the diagnostic and treatment methods disclosed herein can be conjugated to a molecule that decreases tumor cell growth. In other embodiments, the disclosed compositions may comprise antibodies, or fragments thereof, coupled to drugs or prodrugs. Still other embodiments of the present invention comprise the use of antibodies, or fragments thereof, conjugated to specific biotoxins or their cytotoxic fragments such as ricin, gelonin, Pseudomonas exotoxin or diphtheria toxin. The selection of which conjugated or unconjugated antibody to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy tumor cells in animal models, and in some cases in humans. Exemplary radioisotopes include: 90Y, 125I, 131I, 123I, 111In, 105Rh, 153Sm, 67Cu, 67Ga, 166Ho, 177Lu, 186Re and 188Re. The radionuclides act by producing ionizing radiation which causes multiple strand breaks in nuclear DNA, leading to cell death. The isotopes used to produce therapeutic conjugates typically produce high energy alpha- or beta-particles which have a short path length. Such radionuclides kill cells to which they are in close proximity, for example neoplastic cells to which the conjugate has attached or has entered. They have little or no effect on non-localized cells. Radionuclides are essentially non-immunogenic.

### V. EXPRESSION OF ANTI-GITR ANTIBODIES, OR ANTIGEN BINDING FRAGMENTS THEREOF

Following manipulation of the isolated genetic material to provide anti-GITR antibodies of the invention as set forth above, the genes are typically inserted in an expression vector for introduction into host cells that may be used to produce the desired quantity of the claimed antibodies, or fragments thereof.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals. In particularly preferred embodiments the cloned variable region genes are inserted into an expression vector along with the heavy and light chain constant region genes (preferably human) synthetic as discussed above.

In other preferred embodiments the anti-GITR antibodies, or fragments thereof, of the invention may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is incorporated herein. Those skilled in the art will appreciate that such expression systems may be used to effectively produce the full range of polypeptides disclosed in the instant application.

More generally, once a vector or DNA sequence encoding an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, the host cell line used for antibody expression is of mammalian origin; those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (Potelligent® Cells) (Biowa, Princeton, N.J.)). In one embodiment NS0 cells may be used. CHO cells are particularly preferred. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-)affinity chromatography.

Genes encoding the anti-GITR antibodies, or fragments thereof, of the invention can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### VI. PHARMACEUTICAL FORMULATIONS AND METHODS OF ADMINISTRATION OF ANTI-GITR ANTIBODIES

In another aspect, the invention provides pharmaceutical compositions comprising an anti-GITR antibody, or fragment thereof.

Methods of preparing and administering antibodies, or fragments thereof, of the invention to a subject are well known to or are readily determined by those skilled in the art. The route of administration of the antibodies, or fragments thereof, of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to autoimmune or neoplastic disorders.

Effective doses of the stabilized antibodies, or fragments thereof, of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

For passive immunization with an antibody of the invention, the dosage may range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention.

Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered may fall within the ranges indicated.

Antibodies, or fragments thereof, of the invention can be administered on multiple occasions. Intervals between single dosages can be, e.g., daily, weekly, monthly or yearly. Intervals can also be irregular as indicated by measuring blood levels of polypeptide or target molecule in the patient. In some methods, dosage is adjusted to achieve a certain plasma antibody or toxin concentration, e.g., 1-1000 ug/ml or 25-300 ug/ml. Alternatively, antibodies, or fragments thereof, can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies. In one embodiment, the antibodies, or fragments thereof, of the invention can be administered in unconjugated form. In another embodiment, the antibodies of the invention can be administered multiple times in conjugated form. In still another embodiment, the antibodies, or fragments thereof, of the invention can be administered in unconjugated form, then in conjugated form, or vice versa.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding a polypeptide of the invention (e.g., in a vector). Doses for nucleic acids encoding polypeptides range from about 10 ng to 1 g, 100 ng to 100 mg, 1 ug to 10 mg, or 30-300 ug DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. Intramuscular injection or intravenous infusions are preferred for administration of an antibody of the invention. In some methods, therapeutic antibodies, or fragments thereof, are injected directly into the cranium. In some methods, antibodies, or fragments thereof, are administered as a sustained release composition or device, such as a Medipad™ device.

Agents of the invention can optionally be administered in combination with other agents that are effective in treating the disorder or condition in need of treatment (e.g., prophylactic or therapeutic). Preferred additional agents are those which are art recognized and are standardly administered for a particular disorder.

Effective single treatment dosages (i.e., therapeutically effective amounts) of 90Y-labeled antibodies of the invention range from between about 5 and about 75 mCi, more preferably between about 10 and about 40 mCi. Effective single treatment non-marrow ablative dosages of 131I-labeled antibodies range from between about 5 and about 70 mCi, more preferably between about 5 and about 40 mCi. Effective single treatment ablative dosages (i.e., may require autologous bone marrow transplantation) of 131I-labeled antibodies range from between about 30 and about 600 mCi, more preferably between about 50 and less than about 500 mCi. In conjunction with a chimeric modified antibody, owing to the longer circulating half life vis-a-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the 111In label, are typically less than about 5 mCi.

While a great deal of clinical experience has been gained with 131I and 0.90Y, other radiolabels are known in the art and have been used for similar purposes. Still other radioisotopes are used for imaging. For example, additional radioisotopes which are compatible with the scope of the instant invention include, but are not limited to, 123I, 125I, 32P, 57Co, 64Cu, 67Cu, 77Br, 81Rb, 81Kr, 87Sr, 113In, 127Cs, 129Cs, 132I, 197Hg, 203Pb, 206Bi, 177Lu, 186Re, 212Pb, 212Bi, 47Sc, 105Rh, 109Pd, 153Sm, 188Re, 199Au, 225Ac, 211A 213Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include 125I, 123I, 99Tc, 43K, 52Fe, 67Ga, 68Ga, as well as 111In. Antibodies have also been labeled with a variety of radionuclides for potential use in targeted immunotherapy (Peirersz et al. Immunol. Cell Biol. 65: 111-125 (1987)). These radionuclides include 188Re and 186Re as well as 199Au and 67Cu to a lesser extent. U.S. Pat. No. 5,460,785 provides additional data regarding such radioisotopes and is incorporated herein by reference.

As previously discussed, the antibodies, or fragments thereof, of the invention, can be administered in a pharmaceutically effective amount for the in vivo treatment of mammalian disorders. In this regard, it will be appreciated that the disclosed antibodies, or fragments thereof, will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of an antibody of the invention, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the polypeptide will preferably be capable of interacting with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the polypeptide.

In keeping with the scope of the present disclosure, the antibodies of the invention may be administered to a human or other animal in accordance with the aforementioned methods of treatment in an amount sufficient to produce a therapeutic or prophylactic effect. The polypeptides of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of polypeptides according to the present invention may prove to be particularly effective.

### VII. METHODS OF TREATING GITR-ASSOCIATED DISEASE OR DISORDERS

The anti-GITR antibodies, or fragments thereof, of the invention are useful for antagonizing GITR activity. Accordingly, in another aspect, the invention provides methods for treating GITR-associated diseases or disorders by administering to a subject in need thereof a pharmaceutical composition comprising one or more anti-GITR antibody, or antigen binding fragment thereof of the invention.

GITR-associated diseases or disorders amenable to treatment include, without limitation, allergic disorders, inflammatory disorders, or autoimmune disorders. Exemplary diseases or disorders include: chronic obstructive pulmonary disease; systemic lupus erythematosus; rheumatoid arthritis; splanchnic artery occlusion shock; spinal cord injury; type 1 Diabetes; multiple sclerosis; allergic bronchopulmonary aspergillosis; Allergic rhinitis; Autoimmune hemolytic anemia; Acanthosis nigricans; Allergic contact dermatitis; Addison's disease; Atopic dermatitis; Alopecia greata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskott-Aldrich syndrome.

In certain embodiments, a pharmaceutical composition comprising one or more anti-GITR antibodies, or antigen binding fragment thereof of the invention is administered to a subject in combination with one or more additional therapeutic agent. In a particular embodiments, the one or more additional therapeutic agents is administered concurrently with the pharmaceutical composition comprising one or more anti-GITR antibodies. Suitable additional therapeutic agents include IL-18 antagonists, IL-12 antagonists, TNF antagonists, methotrexate, corticosteroid, cyclosporin, rapamycin, FK506, and non-steroidal anti-inflammatory agents

One skilled in the art would be able, by routine experimentation, to determine what an effective, non-toxic amount of antibody (or additional therapeutic agent) would be for the purpose of treating a GITR-associated disease or disorder. For example, a therapeutically active amount of a polypeptide may vary according to factors such as the disease stage (e.g., stage I versus stage IV), age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

The present invention is further illustrated by the following examples which should not be construed as further limiting. The contents of Sequence Listing, figures and all references, patents and published patent applications cited throughout this application are expressly incorporated herein by reference.

### EXAMPLES

### Example 1

### Isolation of Anti-GITR Antibodies

Mice were immunized with either the extracellular domain of human GITR or with 300-19 mouse pre-B cells expressing human GITR and hybridomas were generated from the B-cells of mice sero-positive for anti-GITR antibodies. Primary hybridoma screening, performed by ELISA assay using a human GITR-Fc fusion protein and by FACS using GITR-expressing HEK293 cells, identified 70 antibody clones that bound to human GITR. These clones were further analyzed in a competition ELISA for their ability to inhibit the binding of human GITRL to human GITR. This secondary screen identified 22 antibody clones that inhibit the binding of human GITRL to human GITR. Of these 22 antibodies, 21 were also able to bind to cynomolgus monkey GITR in an ELISA assay. The VH, VL and CDR amino sequences of an exemplary antibody (clone 2155) are set above forth in Table 1.

The biological properties of exemplary antibodies generated in accordance with the methods of the invention are set forth in detail below.

### Example 2

### Binding Affinity to the Soluble Extracellular Domain of Human and Cynomolgus Monkey GITR

The binding kinetics of several anti-GITR antibodies to recombinant human GITR (hGITR) and cynomolgus monkey GITR (cGITR) were determined by surface plasmon resonance assay using a BIAcore 2000 (BIAcore Inc., Uppsala, N.J.). Briefly, a CM5 BIAcore biosensor chip was docked into the instrument and activated with 250 μL of 1:1 NHS/EDC at room temperature. A rabbit anti-mouse Fc IgG1 mAb (BIAcore #BR-1005-14) (13.5 μg/mL in 0.05M acetate buffer, pH 5) was immobilized on the activated chips in Flow cell 1. The immobilization was carried out at a flow rate of 5 μL/min. The chip was then blocked by injection of 55 μL of ethanolamine-HCl, pH 8.5, followed by five washes with 50 mM NaOH, 1M NaCl. To measure the binding of anti-GITR mabs to the human GITR protein or cyno GITR protein, antibodies were used at 2 μg/mL in BIAcore running buffer (HBS-EP). Antigens (human GITR or cyno GITR) were injected from 3 to 1000 nM. Following completion of the injection phase, dissociation was monitored in a BIAcore running buffer at the same flow rate for 360 sec. The surface was regenerated between injections using 30 μL of 50 mM NaOH-1 M NaCl. Individual sensorgrams were analyzed using BIAsimulation software. The results of the surface plasmon resonance assays are set forth in Table 3 below.

TABLE 3
Binding affinity of antibodies to the soluble extracellular domain of
hGITR and cGITR as determined by surface plasmon resonance assay.

| | | hGITR-Fc | | cGITR-FC | | Ratio |
|---|---|---|---|---|---|---|
| Name | Batch # | kd (s−1) | KD (M) | kd (s−1) | KD (M) | cKD/hKD |
| 168-GITR1 | LP09049 | 7.61E−04 | 1.00E−08 | No Binding | | — |
| 178--GITR1 | LP09051 | 4.00E−03 | 4.05E−08 | No Binding | | — |
| 244-GITR1 | LP09054 | 1.78E−02 | 1.02E−07 | 3.70E−02 | 2.82E−07 | 2.8 |
| 383-GITR1 | LP09068 | 1.93E−02 | 1.26E−07 | 1.74E−02 | 1.78E−07 | 1.4 |
| 391-GITR1 | LP09069 | 1.60E−02 | 1.30E−07 | 2.63E−02 | 3.69E−07 | 2.8 |
| 698-GITR2 | LP09095 | 2.24E−04 | 6.67E−09 | 7.07E−04 | 3.68E−08 | 5.5 |
| 706-GITR2 | LP09096 | 2.21E−04 | 5.95E−09 | 6.62E−04 | 3.18E−08 | 5.3 |
| 827-GITR2 | LP09097 | 1.77E−04 | 4.43E−09 | 4.73E−04 | 2.09E−08 | 4.7 |
| 954-GITR2 | LP09098 | 1.92E−04 | 4.85E−09 | 4.73E−04 | 2.13E−08 | 4.4 |
| 1362-GITR2 | LP09099 | 7.56E−05 | 1.86E−09 | 2.20E−04 | 9.95E−09 | 5.3 |
| 1649-GITR2 | LP09100 | 7.44E−05 | 1.84E−09 | 2.10E−04 | 9.68E−09 | 5.3 |
| 2155-GITR2 | LP09101 | 5.08E−04 | 9.90E−10 | 1.23E−03 | 2.27E−09 | 2.3 |
| 1718-GITR2 | LP09102 | 3.80E−04 | 2.57E−09 | 3.45E−04 | 3.35E−09 | 1.3 |

### Example 3

### Binding Affinity to the Human GITR-expressing HEK293 Cells

The binding kinetics of anti-GITR antibodies to hGITR expressed on the surface of HEK293 cells were determined by Meso Scale assay. HEK293-Trex-hGITR cells (#2404) were coated at 25000 cells/well on a 96-well High Bind plate (MSD L15XB-3) for 2 hours at room temperature. Wells were washed 2 times with 150 μl/well of PBS and blocked with 150 μl/well of blocking solution for 30 min at room temperature. Following two washes with 150 μl/well of PBS, 25 μL/well of Sulfo-conjugated antibodies were added in 2-fold serial dilutions starting at 100 μg/ml up to 12 dilutions in assay diluent and incubated for 45 min at room temperature followed by two washes with PBS. The antibody binding was evaluated by adding 150 μl/well 1× Read buffer T without surfactant and read using the Mesoscale instrument. The results of the binding assays are set forth in Table 4 below. All antibodies tested exhibited high affinity binding to GITR-expressing HEK293 cells.

TABLE 4
Binding affinity of antibodies to hGITR expressing HEK293 cells.

| hGITR mAb | KD Biacore (M) | KD Meso Scale (M) |
|---|---|---|
| 698 GITR-2 | 6.67E−09 | 6.80E−09 |
| 706 GITR-2 | 5.95E−09 | 7.24E−09 |
| 827 GITR-2 | 4.43E−09 | 4.44E−09 |
| 954 GITR-2 | 4.85E−09 | 4.34E−09 |
| 1649 GITR-2 | 1.84E−09 | 5.38E−09 |
| 1718 GITR-2 | 2.57E−09 | 8.57E−10 |
| 2155 GITR-2 | 9.90E−10 | 3.50E−10 |

### Example 4

### Inhibition of the GITRL Binding to GITR

The ability of anti-GITR antibodies to inhibit the binding of human GITRL to hGITR was assessed using a competition ELISA. Ninety-six well plates were coated with GITR-Ligand protein at 0.5 μg/well at 4° C., overnight. Wells were washed with washing buffer (PBS pH 7.4+0.05 Tween 20) and blocked with 200 μL of blocking buffer (PBS pH 7.4+0.05% Tween 20+1% BSA) for 1 hour at room temperature. Wells were then washed two times with washing buffer and exposed to 100 μL of pre-incubated mixtures of GITR-Fc at 0.25 μg/mL plus anti-GITR antibody in 2-fold serial dilutions starting at 10 μg/ml up to 12 dilutions for 1 hour at room temperature with agitation. Plates were washed four times with washing buffer and GITR-Ligand-bound recombinant human GITR-Fc was detected with the use of peroxidase-conjugated goat anti-human IgG-Fc (Sigma), followed by incubation with the TMB substrate (Interchim)

The results of the binding assays are set forth in Table 5 below. All antibodies generated using the methods disclosed herein were able to antagonize the binding of GITRL to GITR more potently than the prior art MAB689 antibody.

TABLE 5
Inhibition of GITRL binding to GITR by
anti-GITR antibodies of the invention.

| GITR mAb | IC50 (nM) | IC50 (CV) |
|---|---|---|
| 706 GITR-2 | 2.82 | 15% |
| 698 GITR-2 | 1.90 | 9.4% |
| 827 GITR-2 | 2.50 | 12% |
| 954 GITR-2 | 2.18 | 8.3% |
| 1362 GITR-2 | 3.81 | 13% |
| 1649 GITR-2 | 3.76 | 15% |
| 1718 GITR-2 | 1.02 | 4.3% |
| 2155 GITR-2 | 0.96 | 4.3% |
| MAB689 | 13.9 | 7.8% |
| (R&D Systems) | | |

### Example 5

### Cross-Competition Binding Analysis

The ability of anti-GITR antibodies to inhibit the binding of human GITRL to hGITR was assessed using a competition SPR assay. Specifically, antibody clones were tested for their ability to compete with each other for binding to hGITR-Fc captured on immobilized anti-human IgG-Fc on a Biacore™ CM5 chip. The results of the assays are set forth in Table 6. The data shows that clones 1718 and 2155 target the same epitope, while clones 698, 706, 954 and 1649 compete with each other but bind a different epitope than clones 1718 and 2155.

TABLE 6
Cross-competition binding analysis of
anti-GITR antibodies of the invention.

| | MAB689 | 2155 | 1718 | 698 | 706 | 954 | 1649 |
|---|---|---|---|---|---|---|---|
| MAB689 | | + | | | | | |
| 2155 | | | + | X | X | X | X |
| 1718 | | | | | | | X |
| 698 | | | | | | | + |
| 706 | | | | | | | + |
| 954 | | | | | | | + |
| 1649 | | | | | | | |

### Example 6

### NFkB Reporter Assay

The ability of antibody clones 1718 and 2155 to inhibit NFkB signal transduction was tested. Specifically, HEK 293 cells were separately transfected with plasmids expressing human GITR or human GITRL to generate GITR and GITRL expressing cells. The GITR expressing cells were also co-transfected with the NFkB luciferase reporter plasmid, pNFkB-luc (BD Biosciences Clontech, Palo Alto, Calif., USA) and the Renilla luciferase reporter plasmid, pRL-TK (Promega, Madison, Wis., USA). The GITR and GITRL expressing cells were contacted in the presence and absence of antibody clones 1718 and 2155 and the commercial anti GITR antibody, MAB689, (R&D systems) and a luciferase assay performed to determine the effect of each antibody on GITR/GITRL-induced NFkB signal transduction.

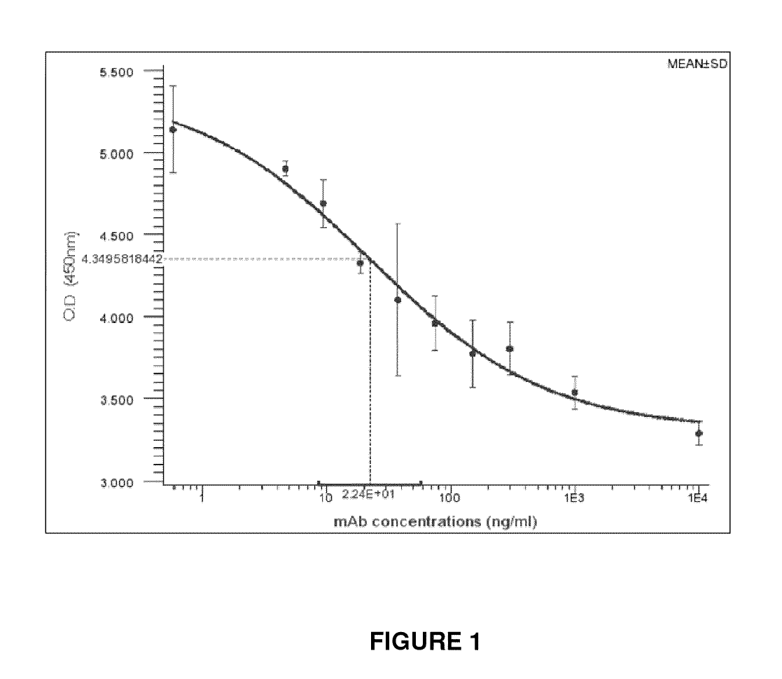

Clone 2155, clone 1718 and MAB689 elicited a 43%, 69% and 18% inhibition of NFkB signal transduction, respectively, when used at a concentration of 300 ng/ml. Thus, the antibodies of the invention are at least 2 fold more potent than the prior art MAB689 at inhibiting NFkB signal transduction. Moreover, as shown in FIG. 1, clone 2155 shows a dose-dependent inhibition of NFkB signal transduction with an IC50 of 0.15 nM.

### Example 7

### Human and Cyomologus Monkey PBMC Binding Assay

The ability of antibody clones 178, 1718 and 2155 to bind to human or cynomolgus monkey GITR on peripheral blood mononuclear cells (PBMCs) was assessed. Specifically, human or cynomolgus monkey peripheral blood mononuclear cells were isloated and diluted to 2×106/ml in RPMI-1640 medium containing 10% FBS. The cells were then stimulated with 5 ug/ml phytohaemagglutinin (PHA) for 72 hours in 6 well plates. The stimulated PBMCs (900,000 cells/well) were then transferred to 96 well V bottom plates, washed and stained with Alexa488 labeled variants, antibody clones 178, 1718 and 2155, the commercial anti GITR antibody, mAb689, (R&D systems) and IgG1, IgG2a, IgG2b controls in 3-fold serial dilutions starting at 30 μg/ml up to 8-10 dilutions for 1 hour at 4° C. The cells were then washed 2 times with FACS stain buffer (Becton Dickinson) and then co-stained with 50 ul of CD4-APC for 20 minutes in the dark at 4° C. The cells were then washed again 2 times with FACS stain buffer before being resuspended in 150 μL of FACS stain buffer (Becton Dickinson) and analyzed using a FACS Calibur instrument. from which binding curves were generated. Equilibrium dissociation constants (K_{D}) for each antibody were determined from the binding curves.

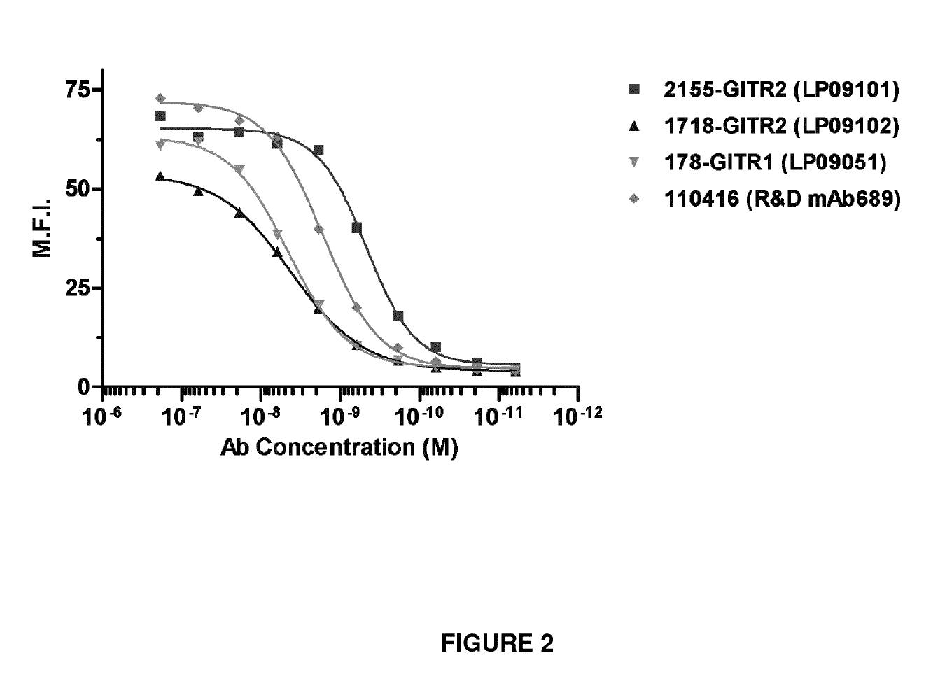

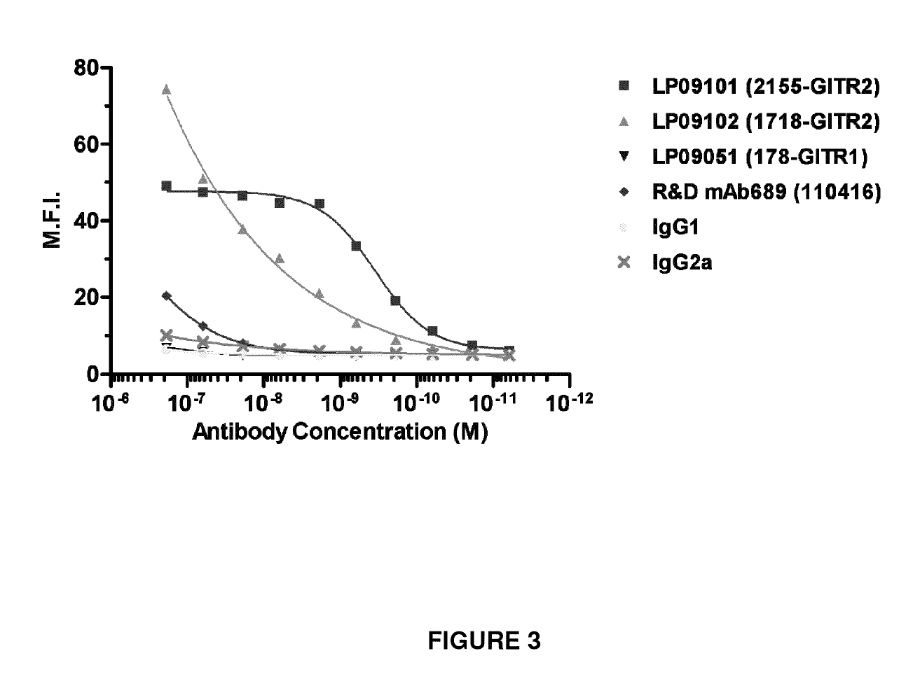

FIG. 2 shows the binding data for human PBMCs. The data shows that antibody clones 178, 1718, 2155, and MAB689 exhibited K_{D}s of 4.5×10⁻⁹M, 4.13×10⁻⁹ M, 4.76×10⁻¹⁰M and 1.66×10⁻⁹M, respectively. FIG. 3 shows the binding data for cynomolgus monkey PBMCs. The data shows that antibody clone 2155 exhibited a K_{D} of 3.53×10⁻¹⁰M, whereas clone 178 and MAB689 did not specifically bind to cynoGITR-expressing PBMCs.

### Example 8

### Humanization of Anti-GITR Antibodies

### a) Humanization Based on Molecular Dynamic Trajectories

The VL and VH amino sequences of the murine 2155 clone were BLASTed in the protein data base (PDB) (Berman et al., Nucleic Acids Research, 2000, 28:235-242) and the closest homologues with equivalent similarity scores were determined. The closest homologues for the VL was 2HRP, 1MF2, and 2HRP, and the closest homologues for the VH was 3BN9, 2DQE and 3DUU. The structures of these homologues were used to build up a homology model of the 2155 variable domains using Molecular Operating Environment (MOE) version 2009.10 (Chemical Computing Group, Quebec, Canada). This model was subsequently energy minimized using standard procedures.

A molecular dynamic (MD) simulation of the 3D homology model of 2155 was subsequently performed for 1.1 nanosecond (ns) in Generalized Born implicit solvent (Gallicchio & Levy, J Comput Chem 2004, 25:479-499). 10 diverse conformations were extracted every 100 picoseconds (ps) for the last 1 ns during this first MD simulation. These 10 diverse conformations are then used as 10 diverse starting points to run 10 molecular dynamic simulations, without constraints on the backbone, at a 300 K temperature for 2.3 ns in Generalized Born implicit solvent. For each of the 10 MD runs, the last 2,000 snapshots, one every ps, from the MD trajectory were then used to calculate, for each murine 2155 amino acid, its root mean square deviations (rmsd) compared to a reference medoid position. By comparing the average rmsd on the 10 separate MD runs of a given amino-acid to the overall average rmsd of all 2155 murine amino-acids, it was decided whether an amino-acid was flexible enough to be considered likely to interact with T-cell receptors and responsible for activation of the immune response. 32 amino-acids were identified as flexible in the murine 2155 antibody, excluding the CDR and its immediate 5 Å vicinity.

The humanizing mutations were identified by determining which human antibody germ line is the most similar to the murine 2155 antibody in terms of their most flexible amino acids. To do so, the motions of the 60 most flexible amino acids of the murine antibody, during the 20 ns (10×2 ns) of molecular dynamic simulation, are compared to the motions of the corresponding amino acids of 49 homology models of human antibody germ lines, for each of which 10 molecular dynamic simulations have been run using the same protocol. Forty nine 3D homology models of human antibody germ lines were built by systematically combining the 7 most frequent human light chain. The humanizing mutations are found by determining which human antibody germ line is the most similar to the murine antibody in terms of their most flexible amino acids. To do so, the motions of the 60 most flexible amino acids of the murine antibody, during the 20 ns (10.times.2 ns) of molecular dynamic simulation, are compared to the motions of the corresponding amino acids of 49 homology models of human antibody germ lines, for each of which 10 molecular dynamic simulations have been run using the same protocol. The 49 3D homology models of human antibody germ lines were built by systematically combining the 7 most frequent human light chain (v.kappa.1, v.kappa.2, v.kappa.3, v.kappa.4, v.lamda.1, v.lamda.2, v.lamda.3) and the 7 most frequent heavy chains (vh1a, vh1b, vh2, vh3, vh4, vh5, vh6) (Nucleic Acids Research, 2005, Vol. 33, Database issue D593-D597). The 60 most flexible amino acids exclude any amino acid in the CDR, and its immediate vicinity, i.e. amino acid with alpha-carbon at a distance of less than 5.ANG. to any a carbon of CDR amino acids as seen in the 3D homology model. (Nucleic Acids Research, 2005, Vol. 33, Database issue D593-D597). The vk1-vh2 human germline antibody showed a 81% 4D similarity of its flexible amino-acids compared to the flexible amino-acids of the murine 2155 antibody and, thus, the vk1-vh2 germline amino acid sequences was used to humanize the 2155 antibody. For pair wise amino-acid association between the murine 2155 vk1-vh2 amino-acids, the 2 sequences were aligned based on the optimal 3D superposition of the alpha carbons of the 2 corresponding homology models.

### b) Stabilizing Mutations

To improve the stability of VL and VH regions of the anti-GITR 2155 antibody, the amino-acids of the VL and VH with low frequency of occurrence vs. their respective canonical sequences, excluding the CDRs, can be mutated into the most frequently found amino-acids at each position (ΔΔGth>0.5 kcal/mol (Monsellier et al. J. Mol. Biol. 2006, 362,580-593). Consensus mutations for the 2155 VL and VH were restricted to the amino-acids found in the closest human germline (i.e vk1-vh2). None of these mutations are located in the “Vernier” zone (Foote et al., J. Mol. Biol. 1992, 224, 487-499). Other criteria were taken into account to consider these consensus mutations for inclusion in the anti-GITR 2155 antibody including favourable changes of hydropathy at the surface or a molecular mechanics based predicted stabilization of the mutant. Stabilizing mutations reported to be successful in the literature (Bedouelle, H. J. Mol. Biol. 2006, 362,580-593; Steipe B. J. Mol. Biol. 1994, 240, 188-192) were also considered. Hydrophobic regions of the antibody were also explicitly identified by analyzing the molecular dynamics simulation of the Fab in a binary solvent (20% isopropanol in water, 20 ns production simulation). Lysine mutations were then introduced in the vicinity of these regions as an attempt to prevent the aggregation.

### c) Humanization by CDR Grafting

The CDRs from the murine anti-GITR 2155 antibody can be grafted onto antibody framework regions comprising fully human sequences. To achieve this, the human antibody germline amino acid sequences with the highest sequence identity to the anti-GITR 2155 VL and VH amino acid sequences were identified by BLAST search. The closest human germlines were identified were: IGKV7-3-01_IGKJ1-01 and IGHV3-53-01_IGHD3-10-02_IGHJ4-01 with respectively 73% 72% identity to the anti-GITR 2155 VL and VH amino acid sequences respectively. Humanizing mutations were obtained by performing a pairwise comparison of the 2 aligned sequences, excluding the CDR (Kabat numbering) & Vernier zone residues. The framework regions of the VH and VL of the murine anti-GITR 2155 antibody were then altered to incorporate the identified humanizing mutations.

### d) Removal of Unwanted Sequence Motifs

Unwanted amino acid motifs were searched for in the resulting humanized sequences using a BLAST search of the protein data base (PDB) (Berman et al., Nucleic Acids Research, 2000, 28:235-242). In addition, known B-cell or T-cell epitopes were searched for in the resulting humanized sequences using the Immune Epitope Data Base (IEDB) (PLos Biol (2005) 3(3)e91). The following sequence motifs were also specifically considered: Asp-Pro (acid labile bond), Asn-X-Ser/Thr (glycosylation, X=any amino-acid but Pro), Asp-Gly/Ser/Thr (succinimide/iso-asp formation in flexible areas), Asn-Gly/His/Ser/Ala/Cys (exposed deamidation sites), Met (oxidation in exposed area). Any unwanted amino acid sequence motifs were removed by site-directed mutagenesis.

### e) Humanized VH and VL Regions

Four humanized versions for the anti-GITR 2155 antibody VL (LC1, LC2a, LC2b, LC3) and five versions anti-GITR 2155 antibody VH (HC1, HC2, HC3a, HC3b, HC4) were generated. The particular combination of amino acid residues altered in each humanized 2155 VL and VH variant are set forth in Table 7 and Table 8 below, respectively. The complete amino acid sequences of the humanized VH and VL domains are set forth in Table 2 above.

TABLE 7
Mutations in the VL variants of anti-GITR 2155
antibody. Stabilizing mutations are underlined.

| | (LC1) | (LC2a) | (LC2b) | (LC3) |
|---|---|---|---|---|
| A12 | | S | S | |
| V13 | A | A | A | |
| L15 | V | V | V | P |
| Q17 | D | D | D | |
| S22 | | | | T |
| F36 | | | Y | |
| Q46 | K | | | |
| S47 | | | | P |
| S76 | | | | T |
| N78 | | T | T | T |
| H80 | | S | S | N |
| M82 | | | | V |
| E83 | Q | Q | Q | |
| E84 | P | P | P | A |
| M89 | T | T | T | N |
| F91 | | Y | Y | Y |
| G104 | | | | Q |
| L109 | | | | V |
| | 7 | 10 | 11 | 12 |
| | mutations | mutations | mutations | mutations |

TABLE 8
Mutations in the VH variants of anti-GITR 2155 antibody.
Stabilizing mutations are underlined. Problematic
motif residues are marked with an asterisk.

| | (HC1) | (HC2) | (HC3a) | (HC3b) | (HC4) |
|---|---|---|---|---|---|
| E1 | Q | Q | Q | Q | |
| K3 | T | T | T | T | Q |
| V5 | | | | K | |
| V12 | | | | | I |
| K13 | | | | | Q |
| K19 | T | T | T | T | |
| G23 | | | | | A |
| S40 | | | | | A |
| E42 | G | G | G | G | G |
| R44 | A | A | A | A | G |
| P60 | | | | | A |
| S62 | | | K | K | |
| V63 | | | F | F | |
| R64 | | | | | K |
| A74 | | | | | S |
| R75 | K | K | K | K | K |
| S77 | | | | | T |
| Q81 | T | T | T | T | |
| S83 | | | | | N |
| R86 | D | D | | | |
| S87 | | | | | A |
| E88 | V | V | | | |
| M92 | | T* | T* | T* | V |
| | 9 | 10 | 10 | 11 | 15 |
| | mutations | mutations | mutations | mutations | mutations |

The LC1 variant displays 7 mutations which derive from the direct comparison between the non-CDR most flexible amino-acids of the anti-GITR 2155 light chain and the vk1 human germline light chain. The LC2a variant includes potentially stabilizing mutations. Stabilizing mutations (Bedouelle, H. J. Mol. Biol. 2006, 362,580-593) include N78T (“L3 variant in the article”), H80S (“L4 variant”) & F91Y (“L5 variant”). Q46 is back-mutated as compared to LC1 (“L1 variant”). A12S was identified by the consensus analysis & is changing favourably the hydropathy at the surface. LC2a displays 10 mutations in total. The LC2b variant derives from LC2a with the introduction of a stabilizing mutation (Steipe B. J. Mol. Biol. 1994, 240, 188-192). Since it is located in the CDRs, this variant is proposed with a lesser priority since the impact on the affinity to the antigen is unpredictable. LC2b displays 11 mutations in total. The LC3 version displays 13 mutations which derive from the grafting method.

The HC1 variant displays 9 mutations which derive from the direct comparison between the non-CDR most flexible amino-acids of the anti-GITR 2155 heavy chain and the vh2 human germline heavy chain. The HC2 variant includes one mutation to address a potentially problematic amino-acid (M92T). HC2 displays 10 mutations in total. The HC3a variant derives from HC2 with the introduction of potentially stabilizing mutations. Stabilizing mutations (Bedouelle, H. J. Mol. Biol. 2006, 362,580-593) include S62K and V63F (“H2 variant in the article. R86 & E88 were back-mutated to reproduce a salt-bridge interaction observed in crystal structures of close human homologs. HC3a displays 10 mutations in total. The HC3b derives from HC3a with the introduction of a LYS (V15K) introducing a favourable change of hydropathy at the surface. The HC4 version displays 15 mutations which derive from the grafting method.

The humanized anti-GITR 2155 antibody VL and VH domains were combined as follows: LC1 and HC1; LC1 and HC2; LC2a and HC3a; LC2a and HC3b; LC2b and HC3a; LC2b and HC3b; and LC3 and HC4.

### Example 9

### Binding Analysis of Humanized Anti-GITR Antibodies

Full length humanized and chimeric variants of anti-GITR antibody 2155 comprising a human IgG4 heavy constant domain and a Ckappa heavy constant domain were produced by transient expression in suspension HEK293 cells. The expressed antibodies were subsequently purified by protein A chromatography and the GITR binding characteristics of the antibodies analyzed.

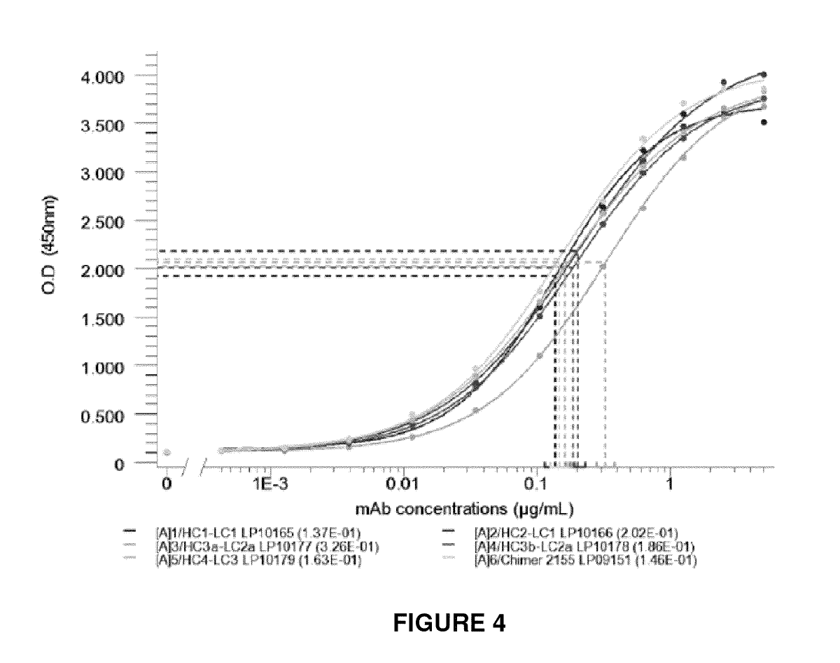

The binding of humanized and chimeric variants of anti-GITR antibody 2155 to hGITR was assessed using an ELISA assay. Ninety-six well plates were coated with GITR protein at 0.25 μg/well at 4° C., overnight. Wells were washed with washing buffer (PBS pH 7.4+0.05 Tween 20) and blocked with 200 μL of blocking buffer (PBS pH 7.4+0.05% Tween 20+1% BSA) for 1 hour at room temperature. Wells were then washed two times with washing buffer and exposed to 100 μL of humanized anti-GITR antibody 2155 variants in 2-fold serial dilutions starting at 5 μg/ml up to 12 dilutions. The plate was incubated at room temperature for 1 h and washed five times with PBS-T. Then, 100 μL of a 1:50 000 dilution of peroxidase-conjugated goat anti-human IgG-Fc (Sigma)) was added to each well. Following incubation at room temperature for 1 h in the dark, the plate was washed with PBS-T, five times. Antibody binding was visualized by adding 100 μL/well of TMB-H₂O₂ substrate buffer (Interchim) The reaction proceeded at room temperature for 10 min and was read using an ELISA plate reader at a wavelength of 450 nm. The binding data, set forth in FIG. 4 and Table 9, shows that all humanized variants of antibody 2155 bind to hGITR with similar binding characteristics as the chimeric 2155 antibody.

TABLE 9
Results of ELISA binding assay measuring binding of humanized
and chimeric variants of anti-GITR antibody 2155 to hGITR.

| Antibody | EC50 | 95% CI | CV (%) |
|---|---|---|---|
| HC1-LC1 | 0.137 (0.91 nM) | [0.114; 0.166] | 8.2 |
| HC2-LC1 | 0.202 (1.34 nM) | [0.177; 0.231] | 5.7 |
| HC4-LC3 | 0.163 (1.08 nM) | [0.147; 0.180] | 4.4 |
| HC3a-LC2a | 0.326 (2.17 nM) | [0.279; 0.382] | 6.8 |
| HC3b-LC2a | 0.186 (1.24 nM) | [0.182; 0.191] | 1.1 |
| Chimeric 2155 | 0.146 (0.97 nM) | [0.125; 0.170] | 6.6 |

The ability of the humanized and chimeric variants of anti-GITR antibody 2155 to antagonize the binding of hGTRL to hGITR was assessed using a competition ELISA assay. Ninety-six well plates were coated with GITR-Ligand proteins at 0.5 μg/well at 4° C. overnight. Wells were washed with washing buffer (PBS pH 7.4+0.05 Tween 20) and blocked with 200 μL of blocking buffer (PBS pH 7.4+0.05% Tween 20+1% BSA) for 1 hour at room temperature. Wells were then washed two times with washing buffer and exposed to 100 μL of pre-incubated mixtures of GITR at 0.25 μg/mL plus humanized anti-GITR antibody 2155 variants in 2-fold serial dilutions starting at 20 μg/ml up to 12 dilutions for 1 hour at room temperature with agitation. Plates were washed four times with washing buffer and GITR-Ligand-bound recombinant human GITR-Fc was detected with the use of peroxidase-conjugated goat, anti-human IgG-Fc (Sigma), followed by incubation with the TMB substrate (Interchim ref UP664780).

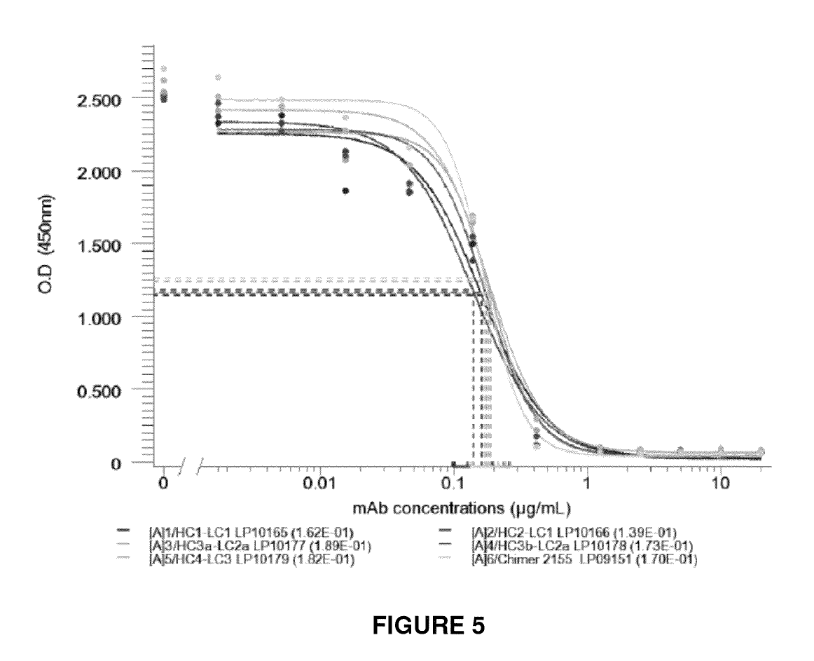

The binding data, set forth in FIG. 5 and Table 10, shows that inhibition of hGITRL binding to hGITR by all of the humanized variants of antibody 2155 was similar to that of the chimeric 2155 antibody.

TABLE 10
Results of competition ELISA assay measuring inhibition
of binding of GITRL to hGITR by humanized and chimeric
variants of anti-GITR antibody 2155.

| Antibody | IC50 | 95% CI | CV (%) |
|---|---|---|---|
| HC1-LC1 | 0.162 (1.08 nM) | [0.101; 0.261] | 21.0 |
| HC2-LC1 | 0.139 (0.93 nM) | [0.098; 0.197] | 15.0 |
| HC4-LC3 | 0.182 (1.21 nM) | [0.138; 0.240] | 12.0 |
| HC3a-LC2a | 0.189 (1.26 nM) | [0.135; 0.264] | 15.0 |
| HC3b-LC2a | 0.173 (1.15 nM) | [0.126; 0.236] | 14.0 |
| Chimeric 2155 | 0.170 (1.13 nM) | [0.134; 0.216] | 10.0 |

The ability of the humanized and chimeric variants of anti-GITR antibody 2155 to bind to GITR-expressing HEK293 cells was assessed using a FACS-based binding assay. HEK293-Trex-hGITR cells (#2404) were plated at 50000 cells/well on a 96-well plate and stained with humanized anti-GITR antibody 2155 variants in 2-fold serial dilutions starting at 30 μg/ml up to 12 dilutions for 1 hour at 4° C. Wells were washed 3 times with 200 μl/well of PBS+1% BSA by centrifugation at 350 g for 5 minutes. Then, 100 μL of a 1:400 dilution of Alexa 647-conjugated, anti-human IgG-Fc (Invitrogen) was added to each well. Following incubation at 4° C. for 45 minutes in the dark, the plate was washed with PBS-1% BSA three times. Cells were resuspended in 150 μL of PBS-1% BSA and analyzed using GUAVA instrument.

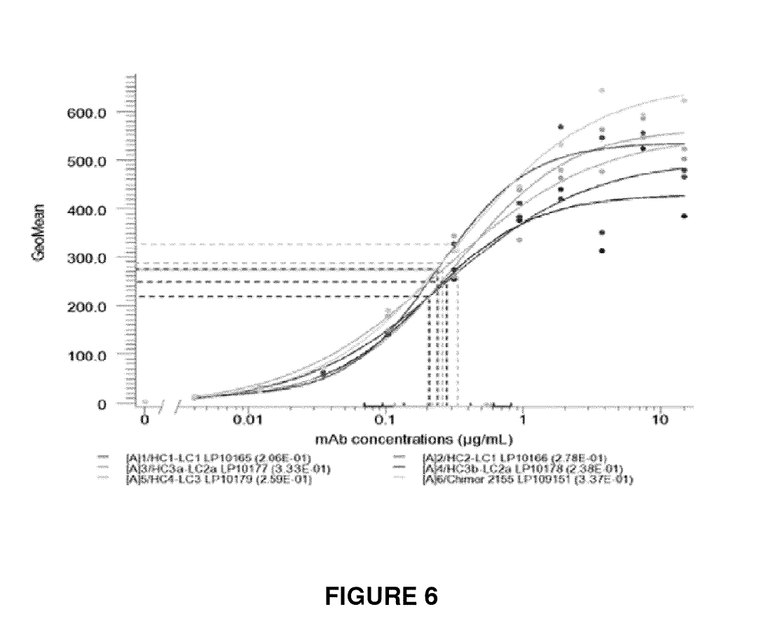

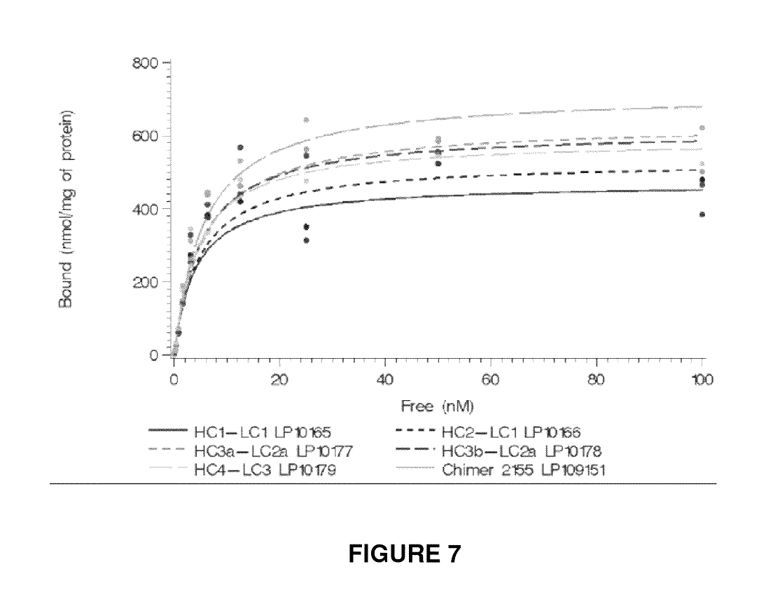

The binding data, set forth in FIGS. 6 and 7 and Tables 11 and 12, shows that all humanized variants of antibody 2155 exhibited similar binding affinities for cell surface hGITR to that of the chimeric 2155 antibody.

TABLE 11
Results of FACS binding assay measuring binding
of humanized and chimeric variants of anti-GITR
antibody 2155 to GITR-expressing HEK-293 cells.

| Antibody | EC50 | 95% CI | CV (%) |
|---|---|---|---|
| HC1-LC1 | 0.206 | [0.069; 0.612] | 49 |
| HC2-LC1 | 0.278 | [0.094; 0.816] | 48 |
| HC4-LC3 | 0.259 | [0.116; 0.578] | 35 |
| HC3a-LC2a | 0.333 | [0.203; 0.547] | 21 |
| HC3b-LC2a | 0.238 | [0.135; 0.417] | 24 |
| Chimeric 2155 | 0.337 | [0.219; 0.520] | 18 |

TABLE 12
Results of FACS binding assay measuring binding of humanized and
chimeric variants of anti-GITR antibody 2155 to GITR-expressing
HEK-293 cells. Data was generated from a Michaelis binding plot.

| Antibody | KD (nM) | CV (%) |
|---|---|---|
| HC1-LC1 | 4.03 | 28.6 |
| HC2-LC1 | 4.68 | 23.9 |
| HC4-LC3 | 4.52 | 19.7 |
| HC3a-LC2a | 5.51 | 20.4 |
| HC3b-LC2a | 4.96 | 26.3 |
| Chimeric 2155 | 5.55 | 19.3 |

### Example 10

### Anti-GITR Antibodies Prevent Airway Hyperresponsiveness in Cynomolgus Monkeys

Pulmonary function was monitored using a respiratory analyzer (Modular Instruments Inc). The acute bronchoconstriction was measured by change in lung resistance before and after antigen challenge. Airway hyperresponsiveness was measured by performing a dose response curve to inhaled methylcholine. Airway inflammation was assessed by performing a bronchoalveolar lavage and measuring total cell numbers and cell differentials, using the Advia 120 hematology analyzer.

### Baseline:

Animals were anesthetized with ketamine/medazolam or ketamine/medetomidine, IM, while in their cage. Once sedated, they were brought into the laboratory and the pre-treatment blood samples were drawn from the femoral vein or vascular access port (VAP), 4-16 ml in EDTA and/or serum tubes. EDTA samples were counted using the Advia for cell differentials and total cell counts. The samples may be kept on ice until processed (plasma and/or serum separated). Baseline AHR and airway inflammation were then assessed.

Animals were intubated, ventilated and seated in a specially adapted chair and connected to the MI2 respiratory analyzer. The analyzer uses the primary signals of airflow and transthoracic pressure to compute lung resistance and compliance. Once a stable lung resistance baseline was established animals were challenged with inhaled phosphate buffered saline (PBS) and the lung resistance monitored for 10 minutes. Following this the animals were challenged with increasing doses of inhaled methylcholine (0.1-30 mg/ml), followed by ten minutes monitoring, until either the PBS-baseline lung resistance had doubled, or the final dose of methylcholine was administered. The PC100 (dose of methylcholine required to cause a 100% increase in baseline lung resistance) was interpolated from a plot of methylcholine dose against percent increase in lung resistance. Following the conclusion of the dose response curve, the animal's lungs were lavaged with 2×10 ml aliquots of normal saline using a pediatric bronchoscope inserted down the endotracheal tube. The recovered lavage fluid was stored on ice until processed for total cell counts, cell differentials (using the Advia) and cells were frozen (−80° C.) for future analysis.

Animals were then reversed with flumazenil IV or antisedan IM, recovered in an incubator and returned to their home cage.

### Day −7

Animals were anesthetized with ketamine/metatomidine IM. Once in the laboratory animals were dosed with SAR258650 (LP09169, formulation=protein 9.78 mg/ml) 2.5 mg/kg or Dulbecco's phosphate buffered saline 1 ml/kg intravenously. Animals were monitored closely for any adverse reactions. Once dosed, animals were reversed with antisedan 1M, recovered and returned to their home cage and monitored throughout the day.

### Day 0

Animals were anesthetized as day −7. The animals were then intubated, a blood sample was taken from the femoral vein, (4-8 ml in EDTA and/or serum tubes which may be placed on ice) then placed onto a mechanical ventilator with supplemental 1-2% isoflurane and connected to the MI2 respiratory analyzer as above. After establishing a stable baseline measurement for lung resistance, animals were challenged with inhaled Ascaris suum antigen, delivered for 20-30 secs. The Ascaris concentration was previously titrated for each animal, to give an increase in lung resistance exceeding 50% above baseline. Following the antigen challenge, the lung resistance was monitored for 10 minutes, and the maximum increase over baseline was noted Animals were then reversed, recovered and returned to their home cage.

### Day +1

Animals were anesthetized with ketamine/medazolam or etamine/medatomidine, IM, intubated and connected to the MI2 respiratory analyzer. AHR and airway inflammation was measured using the same methods as the Baseline. Blood samples were drawn from the femoral vein or vascular access port (VAP), 4-16 ml in EDTA and/or serum tubes. EDTA samples were counted using the Advia for cell differentials and total cell counts. The difference in PC100, and BAL cells between Baseline and Day +1 were used to assess the efficacy of the drug treatments.

There were 2 arms to this study. A minimum “wash out” period of at least 10 weeks was implemented between each arm of study.

### Endpoint Measurements:

1. AHR, Acute bronchoconstriction, BAL cells

2. Plasma antibody levels

Baseline: BAL (cells & supernatant), Blood (CBC+plasma cytokines), Serum (PK)
Day 0: Blood (serum-PK)
Day 1: BAL (cells & supernatant), Blood (CBC+plasma cytokines)

1) An isolated monoclonal antibody, or antigen binding portion thereof, that binds specifically to human GITR comprising an HCDR3 region amino acid sequence selected from the group consisting of SEQ ID NO: 1, 18, 29, and 35, or conservative amino acid substitutions thereof.

2) The antibody, or antigen binding portion thereof, of claim 1, further comprising an HCDR2 region amino acid sequence selected from the group consisting of SEQ ID NO: 2, 19, 30, 33, and 36, or conservative amino acid substitutions thereof.

3) The antibody, or antigen binding portion thereof, of claim 2, further comprising an HCDR1 region amino acid sequence selected from the group consisting of SEQ ID NO: 3, 20, 37 and 43, or conservative amino acid substitutions thereof.

4) The antibody, or antigen binding portion thereof, of claim 2, further comprising an LCDR3 region amino acid sequence selected from the group consisting of SEQ ID NO: 4, 21, 26, and 38, or conservative amino acid substitutions thereof.

5) The antibody, or antigen binding portion thereof, of claim 2, further comprising an LCDR2 region amino acid sequence selected from the group consisting of SEQ ID NO: 5, 22, and 38, or conservative amino acid substitutions thereof.

6) The antibody, or antigen binding portion thereof, of claim 2, further comprising an LCDR1 region amino acid sequence selected from the group consisting of SEQ ID NO: 6, 23, and 40, or conservative amino acid substitutions thereof.

7) An isolated monoclonal antibody, or antigen binding portion thereof, that binds specifically to human GITR comprising an LCDR3 region amino acid sequence selected from the group consisting of SEQ ID NO: 4, 21, 26, and 38, or conservative amino acid substitutions thereof.

8) The antibody, or antigen binding portion thereof, of claim 7, further comprising an LCDR2 region amino acid sequence selected from the group consisting of SEQ ID NO: 5, 22, and 38, or conservative amino acid substitutions thereof.

9) The antibody, or antigen binding portion thereof, of claim 8, further comprising an LCDR1 region amino acid sequence selected from the group consisting of SEQ ID NO: 6, 23, and 40, or conservative amino acid substitutions thereof.

10) An isolated monoclonal antibody, or antigen binding portion thereof, that binds specifically to human GITR comprising a heavy chain variable region amino acid sequence with at least 90% identity to an amino acid sequence selected from the group consisting of SEQ ID NOs: 7, 9, 10, 11, 12, 13, 24, 27, 31, 34, and 41.

11) The antibody, or antigen binding portion thereof, of claim 10, further comprising a light chain variable region amino acid sequence with at least 90% identity to an amino acid sequence selected from the group consisting of SEQ ID NOs: 8, 14, 15, 16, 17, 25, 28, 32, and 42.

12) An isolated monoclonal antibody, or antigen binding portion thereof, that binds specifically to human GITR comprising a light chain variable region amino acid with at least 90% identity to an amino acid sequence selected from the group consisting of SEQ ID NOs: 8, 14, 15, 16, 17, 25, 28, 32, and 42.

13) An isolated monoclonal antibody, or antigen binding portion thereof, that binds specifically to human GITR comprising the heavy chain and light chain variable region amino acid sequences set forth in SEQ ID NO: 7 and 8, SEQ ID NO: 24 and 25, SEQ ID NO: 27 and 28, SEQ ID NO: 31 and 32, SEQ ID NO: 34 and 32, or SEQ ID NO: 41 and 42, respectively.

14) The antibody, or antigen binding portion thereof, of claim 1, further comprising one or more amino acid substitution at positions selected from the group consisting of H1, H3, H5, H12, H13, H19, H23, H40, H42, H44, H60, H62, H63, H64, H74, H75, H77, H81, H83, H86, H87, H88, and H92.

15) The antibody, or antigen binding portion thereof, claim 1, further comprising one or more amino acid substitutions at positions selected from the group consisting of L12, L13, L15, L17, L22, L36, L46, L47, L76, L78, L80, L82, L83, L84, L89, L91, L104, and L109.

16) An isolated monoclonal antibody, or antigen binding portion thereof, that binds specifically to human GITR, wherein the antibody, or antigen binding portion thereof, competes for binding to human GITR with an antibody comprising the heavy chain and light chain variable region amino acid sequences set forth in SEQ ID NO: 7 and 8, SEQ ID NO: 24 and 25, SEQ ID NO: 27 and 28, SEQ ID NO: 31 and 32, SEQ ID NO: 34 and 32, or SEQ ID NO: 41 and 42, respectively.

17) The antibody, or antigen binding portion thereof, of claim 1 which binds specifically to cynomolgus monkey GITR.

18) An isolated nucleic acid encoding the amino acid sequence of the antibody, or antigen binding portion thereof, of claim 1.

19) A recombinant expression vector comprising the nucleic acid of claim 18.

20) A host cell comprising the recombinant expression vector of claim 19.

21) A method of producing an antibody that binds specifically to human GITR, comprising culturing the host cell of claim 20 under conditions such that an antibody that binds specifically to human GITR is produced by the host cell.

22) A pharmaceutical composition comprising the antibody, or antigen binding portion thereof, claim 1 and one or more pharmaceutically acceptable carrier.

23) A method for treating a disease or disorder GITR-associated disease or disorder, the method comprising administering to a subject in need of thereof the pharmaceutical composition of claim 22.

24) The method of claim 23, wherein the disease or disorder is an inflammatory or autoimmune disease or disorder.

25) The method of claim 24, wherein the disease or disorder is chronic obstructive pulmonary disease, systemic lupus erythematosus, rheumatoid arthritis, splanchnic artery occlusion shock, spinal cord injury, type 1 Diabetes, or multiple sclerosis.

26) The method of claim 23, wherein the pharmaceutical composition is administered in combination with one or more additional therapeutic agents.

27) The method of claim 26, wherein the one or more additional therapeutic agent is selected from the group consisting of IL-18 antagonist, IL-12 antagonist, TNF antagonist, methotrexate, corticosteroid, cyclosporin, rapamycin, FK506, and non-steroidal anti-inflammatory agents.

28) The method of claim 26, wherein the one or more additional therapeutic agents is administered concurrently with the pharmaceutical composition of claim 22.
